(19) United States
(12) Patent Application Publication
(10) Pub. No.: US 2023/0148252 A1
(43) Pub. Date: May 11, 2023

(54) PROCESS FOR TREATING KERATIN MATERIALS USING A FLAVIN DERIVATIVE, A POLYMERIZABLE MOLECULE AND LIGHT RADIATION

(71) Applicant: L'OREAL, Paris (FR)

(72) Inventors: Andrew GREAVES, AULNAY-SOUS-BOIS (FR); Constance MAGNE, CHEVILLY LA RUE (FR); Michel PHILIPPE, AULNAY-SOUS-BOIS (FR); Nicolas DAUBRESSE, SAINT-OUEN (FR)

(21) Appl. No.: 16/955,638

(22) PCT Filed: Dec. 19, 2018
(86) PCT No.: PCT/EP2018/085886

§ 371 Date: Jun. 18, 2020

(30) Foreign Application Priority Data
Dec. 20, 2017 (FR) 1762637

(51) Int. Cl. A61K 8/60; A61K 8/49; A61K 8/55; A61K 8/37; A61Q 5/06

(52) U.S. Cl. CPC A61K 8/602; A61K 8/4953; A61K 8/55; A61K 8/375; A61Q 5/06; A61K 2800/95; A61K 2800/81; A61K 2800/88

(57) ABSTRACT

The present invention relates to a process for treating keratin materials, especially human keratin fibers, comprising the application of a composition comprising i) at least one flavin derivative and ii) at least one polymerizable molecule, preferably a photopolymerizable molecule, and at least one step of exposing said materials to artificial or natural light radiation, in particular for caring for and/or repairing the keratin materials.

### CROSS REFERENCE TO RELATED APPLICATION

This is a national stage application of PCT/EP2018/085886, filed internationally on Dec. 19, 2018, which claims priority to French Application No. 1762637, filed on Dec. 20, 2017, both of which are incorporated by reference herein in their entirety.

The present invention relates to a process for treating keratin materials, especially human keratin fibers, comprising the application of a composition comprising i) at least one flavin derivative and ii) at least one polymerizable molecule, preferably a photopolymerizable molecule, and at least one step of exposing said materials to artificial or natural light radiation, in particular for caring for and/or repairing the keratin materials.

The hair is damaged and weakened by external atmospheric agents such as pollution and bad weather, and also by mechanical or chemical treatments, such as brushing, combing, dyeing, bleaching, permanent-waving, relaxing and repeated washing. The hair thus becomes damaged and may in the long run become dry, coarse, brittle, dull, split and/or soft or else sensitive to humidity, making the hair unmanageable, often with frizziness, and/or difficult to style in a humid environment, in particular in a very humid environment. The hair is then also sensitive to mechanical constraints such as being gripped with an elastic band, for example to make a ponytail, and retains the gripping mark even after the elastic band has been taken off.

To overcome these drawbacks, it is common practice to make use of hair compositions for conditioning the hair by giving it cosmetic properties and also good shaping.

However, the conditioning effect obtained via these hair treatments fades out rapidly over time, and in particular does not have satisfactory persistence with respect to shampoo washing.

Furthermore, these hair compositions have little or no effect on controlling the volume of keratin fibers and/or holding the hairstyle and/or managing the hair in a humid or even a very humid environment (humidity-resistant shape and volume control). They also have little or no effect on the sensitivity to mechanical constraints such as being gripped with an elastic band.

There is thus a real need to develop processes for treating keratin fibers that are capable of conserving or even improving the quality of the fiber such as the softness, disentangling, smoothness, manageability, volume and strength of the hair, for example by reducing the brittleness of the hair, and doing so in a persistent manner, or else by making the hair less sensitive to mechanical constraints such as gripping, or by allowing control of the volume and frizziness and/or shape of the keratin fibers and also of the hairstyle in a humid environment, in particular a very humid environment.

In addition, it is known that riboflavin can initiate photochemical reactions such as the crosslinking of polysaccharides to form hydrogels (see, for example, WO 2010/083039, J. Biomed. Mater. Res. B. Appl. Biomater., Kim, S.-H., Chu, C.-C., 91(1), 390-400 (2009). Other hydrogels have been prepared as riboflavin “delivery drug” [see, for example, Drug. Discovery Today, Piyush Gupta, Kavita Vermani and Sanjay Garg, Vol. 7, No. 10 (2002)]. The hydrogels tested with riboflavin or derivatives have not been used in the field of cosmetics.

WO2016/126121 discloses a composition for scalp and hair comprising a yeast extract, wherein the yeast extract may comprise vitamin B2 (riboflavin).

There is still a need to develop a process for treating keratin materials, in particular keratin fibers such as the hair, more particularly a process for treating keratin fibers in an efficient and long-lasting manner while at the same time limiting the perceived degradation of the hair, in particular in a way that is persistent with respect to successive shampoo washes and to control the shape of the humidity-resistant keratin fibers.

There is also a need to develop a process for treating keratin materials, in particular keratin fibers, especially human keratin fibers such as the hair, more particularly a keratin fiber treatment process that is efficient and, if possible, long-lasting while at the same time limiting the perceived degradation of the hair, especially the perception of dry, coarse, brittle, dull, split and/or soft hair or else hair that is sensitive to humidity often with frizziness, and/or that is difficult to style in a humid environment, in particular in a very humid environment, and especially by making the hair insensitive to mechanical constraints such as gripping with an elastic band so as, for example, to make a ponytail.

This (these) aim(s) are achieved by the process of the invention, i.e. a process for treating keratin materials, in particular keratin fibers, especially human keratin fibers such as the hair, comprising:
1) A Step of Applying a Composition (A) Comprising:

i) one or more flavin derivatives chosen from the compounds of formula (I) below:

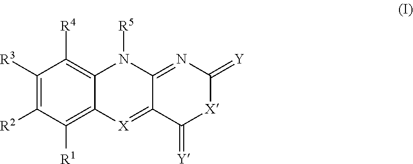

and also the organic or mineral acid or base salts thereof, the optical isomers, geometrical isomers and tautomers thereof, and the solvates thereof such as hydrates; in which formula (I):
R¹, R², R³ and R⁴, which may be identical or different, represent a hydrogen atom, a halogen atom or a group chosen from hydroxyl, (C₁-C₆)alkyl, (C₁-C₆)alkoxy, (C₁-C₆)alkylthio, (di)(C₁-C₆)(alky)amino, nitro(so), in particular chosen from hydrogen and (C₁-C₆)alkyl; more particularly, R¹ and R⁴ represent a hydrogen atom and R² and R³ represent a hydrogen atom or a (C₁-C₄)alkyl group such as methyl;
R⁵ represents a hydrogen atom or a (C₁-C₈)alkyl group optionally substituted with one or more groups chosen from i) hydroxyl, ii) R⁶—C(Y″)—O— with R⁶ representing a hydrogen atom or a (C₁-C₄)alkyl or aryl(C₁-C₄)alkyl group such as benzyl and Y″ represents an oxygen or sulfur atom, or N(R⁷) with R⁷ representing a hydrogen atom or a (C₁-C₄)alkyl group, Y″ preferably representing an oxygen atom, iii) phosphoric (HO)₂P(O)—O—, iv) —O—[(HO)P(O)—O]ₙ-Suc-Het with Suc representing a divalent sugar group such as ribose, and Het representing a heteroaryl group such as adenine, n is an integer equal to 0, 1 or 2, preferably 2; in particular, the group Suc-Het represents a ribose group substituted with an adenine group;
X represents a nitrogen atom or a methylene group C(R⁸) with R⁸ representing a hydrogen atom or a (C₁-C₄)alkyl group; preferably, X represents a nitrogen atom;
X′ represents an oxygen atom or a group NR⁹ with R⁹ representing a hydrogen atom or a (C₁-C₆)alkyl group; in particular, X′ denotes NR⁹, preferably NH;
Y and Y′, which may be identical or different, represent an oxygen or sulfur atom or a group NR¹⁰ with R¹⁰ representing a hydrogen atom or a (C₁-C₆)alkyl group, preferably Y and Y′ represent an oxygen atom; and
ii) one or more polymerizable molecules; and
2) a step of supplying energy to said keratin materials, which consists in exposing said materials to at least one artificial or natural light radiation,
   it being understood that:
   composition (A) may undergo a step of energy supply before being applied to the keratin materials, said energy supply step consisting in exposing said composition to at least one artificial or natural light radiation to lead to a composition (B) which will then be applied to said materials;
   steps 1) and 2) are possibly performed simultaneously with the application, or sequentially, preferably simultaneously with the application or else sequentially to the application 1) and then 2);
   the polymerizable molecule(s) include at least one insaturated group.

The invention also relates to a process for preparing a composition (B), comprising step a) of mixing i) one or more flavin derivatives chosen from the compounds of formula (I) as defined previously, ii) one or more polymerizable molecules as defined below and iii) optionally one or more amines and/or one or more alcohols different i) from the flavins of formula (I), and different ii) from the polymerizable molecules, preferably one or more amines chosen from amino acids; followed by a step b) of exposing said mixture to artificial or natural light radiation.

The invention also relates to a process for preparing a composition (B′), comprising step a) of mixing i) one or more flavin derivatives chosen from the compounds of formula (I) as defined previously, ii) one or more polymerizable molecules chosen from those of formula (II), (IIa), (IIb) or (IIc) as defined below and iii) optionally one or more amines and/or one or more alcohols different i) from the flavins of formula (I), and different ii) from the polymerizable molecules, preferably one or more amines chosen from amino acids; followed by a step b) of exposing said mixture to artificial or natural light radiation.

A subject of the invention is also composition (B) obtained via the process as described previously.

A subject of the invention is also composition (B′) obtained via the process as described previously.

A subject of the invention is also a process for treating keratin materials, in particular keratin fibers, especially human keratin fibers such as the hair, comprising a step of applying to said materials a composition (B) or (B′) obtained via the preparation process as described previously and optionally a step 2) of supplying energy to said materials, consisting in exposing said materials to at least one artificial or natural light radiation, the optional energy supply step 2) being performed simultaneously with the step of applying composition (B) or (B′) or successively to the step of applying composition (B) or (B′) to the keratin materials.

A subject of the invention is also a multi-compartment kit or device:
either comprising in one compartment ingredient i) and in another compartment ingredient ii), or comprising ingredients i) and ii) together in one compartment, and
in another compartment an artificial-light-emitting device.

Another subject of the invention is the cosmetic use i) of one or more compounds of formula (I) as defined previously in the presence ii) of one or more polymerizable molecules and of at least one artificial or natural light radiation; in particular for caring for and/or repairing keratin materials, especially human keratin fibers such as the hair, preferably for repairing the hair.

The processes of the invention use either a composition (A), (B) or (B) containing at least one flavin derivative of formula (I) as defined previously, especially at least one riboflavin phosphate derivative (vitamin B2 derivative), optionally an amino acid such as arginine and at least one polymerizable and especially photocrosslinkable molecule, followed by a step of irradiating with light radiation, and make it possible to improve the cosmetic qualities of keratin fibers. The fibers appear repaired. Fibers treated via one of the processes of the invention after they have been marked or deformed by a mechanical constraint, such as during the gripping of the fibers with a clip, hair slides, or other elastic means such as an elastic band, for example with a ponytail, are improved relative to untreated fibers where the ponytail leaves a long-lasting imprint on the fibers. With one of the processes of the invention, the imprint disappears rapidly, or even immediately.

Other characteristics and advantages of the invention will emerge more clearly on reading the description and the examples that follow.

In the following text, unless indicated otherwise:
the term “salt” means the salts of addition with an organic or mineral acid or base;
the term “organic or mineral acid salt” means cosmetically acceptable addition salts obtained by addition of an organic or mineral acid, more particularly the salts chosen from a salt derived from i) hydrochloric acid HCl, ii) hydrobromic acid HBr, iii) sulfuric acid H₂SO₄, iv) alkylsulfonic acids: Alk-S(O)₂OH such as methylsulfonic acid and ethylsulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; and xv) tetrafluoroboric acid HBF₄;
the term “alkyl” means a linear or branched radical containing from 1 to 12 carbon atoms, in particular from 1 to 8 carbon atoms, more particularly from 1 to 6 carbon atoms, preferably from 1 to 4 carbon atoms, for example methyl, ethyl, n-propyl, isopropyl, butyl, n-pentyl, n-hexyl, preferably methyl or n-propyl and more preferentially methyl;
the term “(C_{X}-C_{y})alkyl” means an alkyl radical as defined previously, said alkyl radical comprising x to y carbon atoms;
the term “alkylene” means a linear or branched divalent radical containing from 1 to 16 carbon atoms, in particular from 1 to 12 carbon atoms, preferably from 1 to 8 carbon atoms, more preferentially from 1 to 6 carbon atoms, even more preferentially from 1 to 4 carbon atoms, for example methylene, ethylene, n-propylene, isopropylene, butylene, n-pentylene or n-hexylene, preferably methylene;
the term “(C_{X}-C_{y})alkylene” means an alkylene radical as defined previously, said alkylene radical comprising x to y carbon atoms;
the term “alkoxy” means an alkyl-oxy group with “alkyl” as defined previously;
the term “(C_{X}-C_{y})alkoxy” means an alkoxy radical as defined previously, said alkoxy radical comprising x to y carbon atoms;
an “aryl” radical represents a monocyclic or polycyclic fused or non-fused carbon-based group, comprising from 6 to 22 carbon atoms, at least one ring of which is aromatic; in particular, the aryl radical is a phenyl, biphenyl, naphthyl, indenyl, anthracenyl or tetrahydronaphthyl, preferably phenyl;
a “heteroaryl radical” represents a 5- to 22-membered, monocyclic or polycyclic, fused or non-fused group, comprising from 1 to 6 heteroatoms chosen from nitrogen, oxygen and sulfur atoms, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, pyrimidyl, pyrimidyl-one, pyrimidyl-dione, tetrazolyl, dihydrothiazolyl, imidazopyridinyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, purinyl-one, purinylepyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl;
the term “sugar” or “Suc” means a monosaccharide group or a disaccharide group, preferably a monosaccharide, each saccharide unit (the saccharide unit in the case of a monosaccharide or each saccharide unit in the case of a disaccharide) comprising one or more hydroxyl groups optionally substituted with a radical R″ chosen from: i) (C₁-C₆)alkyl, (C₂-C₆)alkenyl; ii) an acetyl radical; or iii) a protecting group (PG) for hydroxyl functions, such as (C₂-C₆)alkyl(thio)carbonyl, preferably (C₂-C₆)alkylcarbonyl; in particular R″ represents a (C₁-C₆)alkyl group such as methyl, or an acetyl group; said monosaccharide or disaccharide radical being linked to the rest of the molecule via a bond between the carbon atom C₁ of one of the sugars of said monosaccharide or disaccharide radical, this bond possibly being α or β anomeric; it is understood that, for the compounds of formula (I) as defined previously, when Suc represents a monosaccharide radical, then it is in pyranose form (the sugar heterocycle which constitutes it is 6-membered) or furanose form (the sugar heterocycle which constitutes it is 5-membered); and when Suc represents a disaccharide radical, it comprises a sequence of two identical or different saccharide or oside units which may be in furanose or pyranose form. Preferably, the disaccharide results from the sequence of a saccharide unit in furanose form and a unit in pyranose form or the sequence of a saccharide unit in pyranose form and a unit in furanose form; whether it is for the monosaccharide or polysaccharide radical, each saccharide unit may be in levorotatory L or dextrorotatory D form, and in α or β anomeric form;
the term “polymerizable molecules” means any molecule, commonly referred to as a synthon, which includes at least one reactive group that can react with another synthon to form oligomers or polymers; the reactive group being an unsaturated group, wherein the unsaturated group is preferably —C≡CR′ or —CR═CR′₂ with R and R′, which may be identical or different, representing a hydrogen atom, a (C₁-C₄)alkyl group such as methyl or carboxyl (COOH), preferably a hydrogen atom or a (C₁-C₄)alkyl group such as methyl;
the term “polysaccharides” means a polysaccharide sugar which is a polymer constituted of several saccharides bonded together via O-oside bonds, said polymers being constituted of monosaccharide units as defined previously, said monosaccharide units comprising at least 5 carbon atoms, preferably 6; in particular, the monosaccharide units are linked together via a 1,4 or 1,6 bond as α (alpha) or β (beta) anomer, it being possible for each oside unit to be of L or D configuration, and also the salts thereof and the solvates thereof such as the hydrates of said monosaccharides; more particularly, they are polymers formed from a certain number of saccharides (or monosaccharides) having the general formula: —[Cₓ(H₂O)_{y})]ₙ— where x is an integer greater than or equal to 5, preferably x is greater than or equal to 6, in particular x is between 5 and 7 inclusive and preferably x=6, and y is an integer which represents x−1, and n is an integer greater than or equal to 2, particularly of between 3 and 3000 inclusive, more particularly between 5 and 2500 and preferentially between 10 and 2300;
the term “amino monosaccharide or polysaccharide” means that the monosaccharide or polysaccharide is substituted with one or more amino groups NR₁R₂, i.e. at least one of the hydroxyl groups of at least one saccharide unit is replaced with a group NR₁R₂ with R₁ and R₂, which may be identical or different, representing i) a hydrogen atom, ii) a (C₁-C₆)alkyl group that is optionally substituted, preferably with one or more hydroxyl or NH₂ groups, iii) an aryl group such as phenyl, iv) an aryl(C₁-C₄)alkyl group such as benzyl, v) a (hetero)cyclo(C₅-C₇)alkyl group such as cyclohexyl, morpholinyl, piperazinyl, piperidinyl, vi) a (hetero)cyclo(C₅-C₇)alkyl(C₁-C₄)alkyl group such as cyclohexylmethyl, vii) —C(Y)—(Y′ₚ—R′₁ with Y and Y′, which may be identical or different, representing an oxygen atom, a sulfur atom or N(R′₂), preferably oxygen, p=0 or 1, preferably 0; and R′₁ and R′₂ representing i) to vi) of R₁ and R₂ defined previously, and in particular R′₁ denoting a (C₁-C₆)alkyl group such as methyl. Preferably R₁ and R₂ represent a hydrogen atom or a (C₁-C₄)alkylcarbonyl group such as acetyl;
moreover, the addition salts that may be used in the context of the invention are especially chosen from salts of addition with a cosmetically acceptable base such as the basifying agents as defined below, for instance alkali metal or alkaline-earth metal hydroxides such as sodium hydroxide or potassium hydroxide, ammonia, amines or alkanolamines;
the term “heterocycle” means a 5- to 10-membered monocyclic or bicyclic, preferably monocyclic, fused or non-fused, saturated or unsaturated, aromatic or non-aromatic group, containing from 1 to 3 heteroatoms chosen from nitrogen, oxygen and sulfur atoms, this heterocycle possibly being substituted with one or more radicals, which may be identical or different, chosen from alkyl, hydroxyalkyl and alkoxy radicals; preferentially, according to the present invention, the heterocycle is saturated or unsaturated, preferably saturated, and 5- to 8-membered, and more preferentially is chosen from piperidyl, pyrrolidinyl, piperazinyl and morpholinyl;
the term “cycloalkyl” means a 5- to 10-membered monocyclic or bicyclic, preferably monocyclic, fused or non-fused hydrocarbon-based group which is saturated or contains one or more ethylenic unsaturations, which is non-aromatic, this cycloalkyl possibly being substituted with one or more radicals, which may be identical or different, chosen from alkyl, hydroxyalkyl and alkoxy radicals; preferentially, according to the present invention, the cycloalkyl is saturated and 5- to 8-membered, and more preferentially is chosen from cyclopentyl and cyclohexyl;
the “heterocycle”, “cycloalkyl”, “aryl” and “heteroaryl” radicals may be substituted with at least one substituent borne by a carbon atom or a heteroatom, chosen from:
   an optionally substituted C₁-C₆ alkyl radical;
   a halogen atom;
   a hydroxyl group;
   a C₁-C₂ alkoxy radical;
   a (poly)hydroxy(C₂-C₄)alkoxy radical;
   an amino radical;
   a 5- or 6-membered heterocycloalkyl radical;
   a 5- or 6-membered heteroaryl radical, optionally substituted with a (C₁-C₄)alkyl radical, preferentially methyl;
   an amino radical substituted with one or two identical or different C₁-C₆ alkyl radicals, optionally bearing at least:
i) a hydroxyl group,
ii) an amino group optionally substituted with one or two optionally substituted C₁-C₃alkyl radicals, said alkyl radicals possibly forming with the nitrogen atom to which they are attached a saturated or unsaturated, optionally substituted 5- to 7-membered heterocycle, optionally comprising at least one other nitrogen or non-nitrogen heteroatom,
   an acylamino radical (—NR—C(O)—R′) in which the radical R is a hydrogen atom or a C₁-C₄ alkyl radical optionally bearing at least one hydroxyl group and the R′ radical is a C₁-C₂ alkyl radical;
   a carbamoyl radical ((R)₂N—C(O)—) in which the radicals R, which may be identical or different, represent a hydrogen atom or a C₁-C₄ alkyl radical optionally bearing at least one hydroxyl group;
   an alkylsulfonylamino radical (R′—S(O)₂—N(R)—) in which the radical R represents a hydrogen atom or a C₁-C₄ alkyl radical optionally bearing at least one hydroxyl group and the radical R′ represents a C₁-C₄ alkyl radical or a phenyl radical; an aminosulfonyl radical ((R)₂N—S(O)₂—) in which the radicals R, which may be identical or different, represent a hydrogen atom or a C₁-C₄ alkyl radical optionally bearing at least one hydroxyl group;
   a carboxylic radical in acid or salified (preferably with an alkali metal or a substituted or unsubstituted ammonium) form;
   a cyano group;
   a nitro or nitroso group;
   a polyhaloalkyl group, preferentially trifluoromethyl;
   the term “(di)(Cₓ-C_{y}(alkyl)amino” means an amino radical NH₂, a (Cₓ-C_{y})alkylamino radical, a dialkylamino radical in which each alkyl group is, independently, a C_{X}-C_{y} alkyl radical;
   the term “elastic means” means any means for gripping keratin fibers, in the form of a short yarn, band or strip comprising at least one part made of elastic material such as rubber, of generally circular shape,
   the expression “at least one” is equivalent to “one or more”; and
   the expression “inclusive” for a range of concentrations means that the limits of the range are included in the defined interval.

### i) The Flavins of Formula (I)

Compositions (A), (B) or (B′) of the invention comprise at least one compound of formula (I) as defined previously.

According to a particular embodiment of the invention, the compounds of formula (I) are such that R¹, R², R³ and R⁴, which may be identical or different, represent a hydrogen atom, (C₁-C₆)alkyl, (C₁-C₆)alkoxy or (di)(C₁-C₄)(alkyl)amino, in particular chosen from hydrogen and (C₁-C₄)alkyl. Preferably, R¹ and R⁴ represent a hydrogen atom, and R² and R³ represent a hydrogen atom or a (C₁-C₄)alkyl group such as methyl, preferably a (C₁-C₄)alkyl group such as methyl.

According to a particular embodiment of the invention, the compounds of formula (I) are such that R⁵ represents a (C₁-C₆)alkyl group such as methyl.

According to another preferred embodiment of the invention, the compounds of formula (I) are such that R⁵ represents a (C₁-C₆)alkyl group substituted with one or more identical or different groups chosen from i) hydroxyl, ii) R⁶—C(O)—O— with R⁶ representing a hydrogen atom or a (C₁-C₄)alkyl group such as methyl or n-propyl; iii) phosphoric (HO)₂P(O)—O—, iv) —O—[(HO)P(O)—O]ₙ-Suc-Het with Suc representing a monosaccharide such as ribose, and Het representing an optionally substituted heteroaryl group, in particular optionally substituted pyrimidyl-dione such as adenine, n is an integer equal to 1 or 2, preferably 2; in particular, Suc-Het represents a ribose group, preferably α-D-ribose, optionally substituted with an adenine group.

According to a preferred embodiment, the compounds of formula (I) are such that the radical Suc represents a divalent monosaccharide radical in which the heterocycle constituting it contains 4 or 5 carbon atoms, having the following formula:

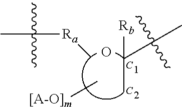

Rₐ representing a (C₁-C₄) alkylene group such as methylene and Rₐ being linked to the rest of the molecule via at least one phosphate group, it being understood that the radical Rₐ is in position C⁵ if the sugar unit is in pyranose form or in position C⁴ if it is in furanose form;
R_{b} represents a hydrogen atom or a group —CH₂—O-A; preferably, R_{b} denotes a hydrogen atom;
A represents a hydrogen atom, a (C₁-C₆)alkyl group or a hydroxy-function-protecting group, such as R_{C}—C(O)— with R_{C} representing a hydrogen atom or a (C₁-C₄)alkyl group such as methyl, or else, when n is greater than or equal to 2 and two groups A-O are contiguous, then two groups A may together form a linear or branched (C₁-C₆)alkylene chain; preferably, all the groups which protect A are identical;
m is equal to 1, 2 or 3; preferably, m=2;

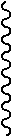

represents the two substitution parts of the divalent monosaccharide.

According to a more particular embodiment, the compounds of formula (I) are such that R⁵ represents the following group:

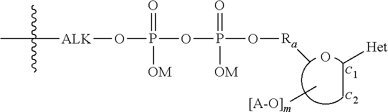

with Rₐ, A, Het and m as defined previously;
ALK representing a (C₁-C₆)alkylene group, preferably pentylene, optionally substituted with one or more hydroxyl groups;
M represents a hydrogen atom or a cationic counterion, in particular an alkali metal or alkaline-earth metal such as Na⁺, K⁺ or ammonium, preferably Na⁺ and

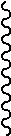

representing the part that is linked to the rest of the molecule.

Preferably, Het represents a purine or pyrimidine nitrogenous base such as adenine, thymine, guanine, cytosine or uracil. Preferably, Het represents an optionally substituted bicyclic heteroaryl group; more particularly, Het represents a purinyl-dione group which is optionally substituted especially with one or more (di)(C₁-C₄)(alkyl)amino groups; preferentially, Het is a purine nitrogenous base such as adenine.

According to a more particular embodiment, the compounds of formula (I) are such that R⁵ represents a (C₁-C₆)alkyl group optionally substituted with one or more hydroxyl or phosphate groups such as pentyl substituted with one, two, three or four hydroxyl groups and optionally with a phosphate group, such as 2,3,4,5-tetrahydroxypentyl or 5-dihydrogenophosphate-2,3,4-trihydroxypentyl.

According to a particular embodiment, the compounds of formula (I) are such that X represents a nitrogen atom.

According to a particular embodiment, the compounds of formula (I) are such that X′ represents a group NR⁹ with R⁹ representing a hydrogen atom or a (C₁-C₆)alkyl group; in particular, X′ preferably denotes NH.

According to a particular embodiment, the compounds of formula (I) are such that Y and Y′ are identical and more particularly represent an oxygen or sulfur atom; preferably, Y and Y′ represent an oxygen atom.

More particularly, the compounds of formula (I) of the invention are chosen from compounds 1 to 7 below:

| Structure | |
|---|---|
| 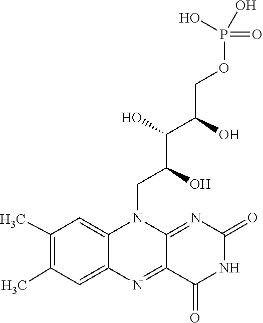 [SMILES: Cc1cc2nc3c(=O)[nH]c(=O)nc-3n(C[C@H](O)[C@H](O)[C@H](O)COP(=O)(O)O)c2cc1C] | 1 |

(2R,3S,4S)-5-(7,8-dimethyl-2,4-dioxo-3,4-
dihydrobenzo[g]pteridin-10(2H)-yl)-

| | |
|---|---|
| 2,3,4-trihydroxypentyl dihydrogen phosphate | |
| or riboflavin 5′-phosphate | |
| 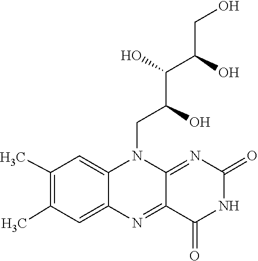 [SMILES: Cc1cc2nc3c(=O)[nH]c(=O)nc-3n(C[C@H](O)[C@H](O)[C@H](O)CO)c2cc1C] | 2 |
| 7,8-dimethyl-10-((2R,3R,4S)-2,3,4,5- | |
| tetrahydroxypentyl)benzo[g]pteridine- | |
| 2,4-(3H,10H)-dione, or vitamin B₂, lactoflavin | |
| 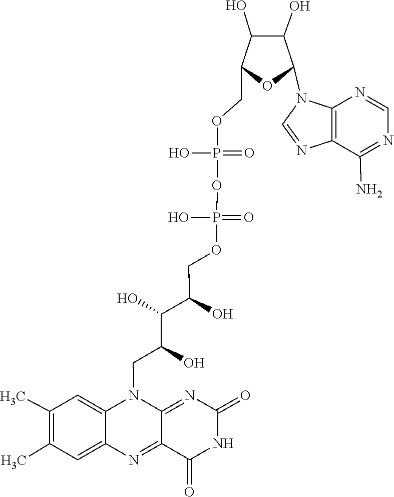 | 3 |
| Flavin adenine dinucleotide (FAD) | |
| 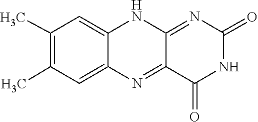 [SMILES: Cc1cc2nc3c(=O)[nH]c(=O)nc-3[nH]c2cc1C] | 4 |
| 7,8-dimethylbenzo[g]pteridine-2,4(3H,10H)-dione | |
| 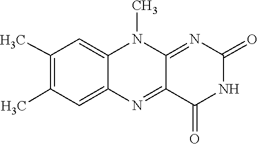 [SMILES: Cc1cc2nc3c(=O)[nH]c(=O)nc-3n(C)c2cc1C] | 5 |
| 7,8,10-trimethylbenzo[g]pteridine-2,4(3H,10H)-dione | |
| 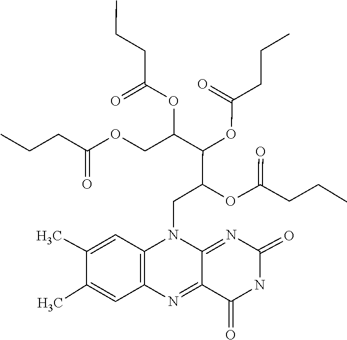 | 6 |
| Riboflavin tetrabutyrate | |
| CAS # 752-56-7 | |
| 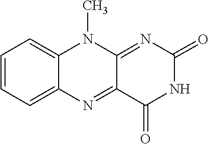 [SMILES: Cn1c2nc(=O)[nH]c(=O)c-2nc2ccccc21] | 7 |
| 10-methylbenzo[g]pteridine-2,4(3H,10H)-dione | |
| CAS = 4074-58-2 | |

and also the organic or mineral acid or base salts thereof, the optical isomers and tautomers thereof, and the solvates thereof such as hydrates, in particular the salt thereof with an alkali metal such as sodium.

According to a particular embodiment, compositions (A), (B) or (B′) comprise one or more compounds of formula (I) as defined previously, in an amount inclusively between 0.01% and 30% by weight relative to the total weight of composition (A), (B) or (B′), in particular between 0.05% and 20%, more particularly between 0.1% and 10%, preferentially between 1% and 5% by weight relative to the total weight of composition (A), (B) or (B′).

### II) The Polymerizable Molecules

According to a particular embodiment of the invention, the polymerizable molecule(s) have a molecular weight of less than or equal to 500 g/mol, preferably a molecular weight inclusively between 26 and 400 g/mol.

Preferably, the polymerizable molecules are photo-polymerizable, i.e. they have the capacity of polymerizing under exposure to at least one natural and/or artificial light radiation.

Preferably, the polymerizable molecule(s) are chosen from those of formula (II) below:

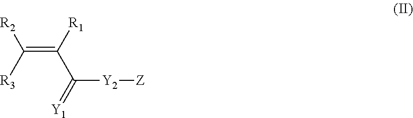

and also the organic or mineral acid or base salts thereof, the optical isomers, geometrical isomers and tautomers thereof, and the solvates thereof such as hydrates; in which formula (II):
R₁ and R₂, which may be identical or different, represent a hydrogen atom or a (C₁-C₈)alkyl or (C₂-C₈)alkenyl group, said alkyl or alkenyl groups being:
   optionally interrupted with one or more heteroatoms such as O, S and N(R₅), with R₅ representing a hydrogen atom or a (C₁-C₄)alkyl group such as methyl, and/or
   optionally substituted with one or more radicals chosen from the following radicals: hydroxyl, (di)(C₁-C₄)(alkyl)amino, (C₁-C₄)alkoxy and —C(Y′₁)-Y′₂—R′₄ such as carboxyl;
R₃ is as defined for R₂, or else represents a group —C(Y′₁)—Y′₂—R′₁
Y₁, Y₂, Y′₁ and Y′₂, which may be identical or different, represent an oxygen or sulfur atom or N(R₅), with R₅ representing a hydrogen atom or a (C₁-C₄)alkyl group, preferably, Y₁ and Y′₁ represent an oxygen atom and Y₂ and Y′₂ represent an oxygen atom or a group N(R₅); more particularly, Y₂ and Y′₂ represent an oxygen atom; and
R₄ and R′₄, which may be identical or different, represent a hydrogen atom or a (C₁-C₁₆)alkyl group optionally substituted with one or more radicals, which may be identical or different, chosen from the following radicals: a) amino (NH₂), mono- or di(C₁-C₄)alkylamino such as dimethylamino, b) hydroxyl (OH), c) SO₃H, d) carboxyl (CO₂H), e) cyano, f) (poly)(halo)(C₁-C₄)alkyl such as trifluoromethyl (CF₃), g) (C₁-C₄)alkoxycarbonyl; said alkyl radical being optionally interrupted with one or more heteroatoms such as O, S and N(R₅) with R₅ as defined previously, and preferably R₅ denoting H or Me;
Z denotes a radical R₄ as defined previously or a radical (Z′) of formula:

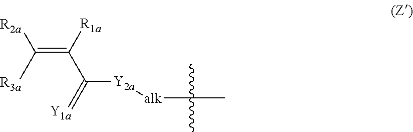

in which formula (Z′):
   Y₁ₐ, Y₂ₐ, R₁ₐ, R₂ₐ and R₃ₐ have, respectively and independently, the same meaning as Y₁, Y₂, R₁, R₂ and R₃ defined previously;
   alk denotes a divalent (C₁-C₁₆)alkylene radical optionally interrupted with one or more oxygen or sulfur atoms or with one or more NH groups, said alkylene radical being optionally substituted with one or more radicals chosen from hydroxyl and a radical of formula (Z″):

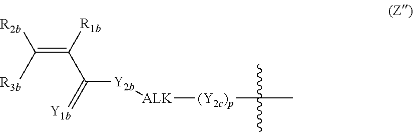

in which formula (Z″):
   Y_{1b}, Y_{2b}, Y_{2c}, R_{1b}, R_{2b} and R_{3b} have, respectively and independently, the same meaning as Y₁, Y₂, R₁, R₂ and R₃ defined previously, ALK denoting a (C₁-C₆)alkylene radical (divalent radical) such as methylene or propylene and p being equal to 0 or 1;

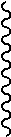

denoting the point of attachment between alk and Y₂ in the case of formula (Z′) and

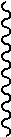

denoting the point of attachment between (Y₂)p and alk in the case of formula (Z″).

According to a particular variant of the invention, Z denotes a radical R₄. According to one embodiment, the polymerizable molecule(s) are of formula (II) in which Z denotes a radical R₄ and R₁ represents a hydrogen atom or a (C₁-C₄)alkyl group. According to one embodiment, the polymerizable molecule(s) are of formula (II) in which Z denotes a radical R₄ and R₁ represents a hydrogen atom. According to another particular embodiment, the polymerizable molecule(s) are of formula (II) in which Z denotes a radical R₄ and R₁ represents a (C₁-C₄)alkyl group such as methyl.

According to another embodiment, the polymerizable molecule(s) are of formula (II) in which Z denotes a radical R₄ and R₁ represents a (C₁-C₄)alkyl group substituted with a radical chosen from hydroxyl and —C(Y′₁)—(Y′₂—R′₄, in particular —C(O)—O—R′₄ with R′₄ as defined previously; preferably R₁ denotes a (C₁-C₄)alkyl group substituted with a carboxyl group such as carboxymethyl.

According to one embodiment, the polymerizable molecule(s) are of formula (II) in which Z denotes a radical R₄ and R₂ represents a hydrogen atom, a (C₁-C₄)alkyl group optionally substituted with one or more radicals chosen from hydroxyl, (di)(C₁-C₄)(alkyl)amino, (C₁-C₄)alkoxy and carboxyl radicals, R₂ preferably represents a hydrogen atom or a (C₁-C₄)alkyl group such as methyl, in particular a hydrogen atom.

According to one embodiment, the polymerizable molecule(s) are of formula (II) in which Z denotes a radical R₄ and R₃ represents i) a hydrogen atom, ii) a (C₁-C₄)alkyl group optionally substituted with one or more radicals chosen from hydroxyl, (di)(C₁-C₄)(alkyl)amino, (C₁-C₄)alkoxy and carboxyl radicals; iii)

According to one embodiment, the polymerizable molecule(s) are of formula (II) in which Z denotes a radical R₄ and R₃ represents i) a hydrogen atom.

According to another embodiment, the polymerizable molecule(s) are of formula (II) in which Z denotes a radical R₄ and R₃ represents a (C₁-C₄)alkyl group optionally substituted with a radical chosen from hydroxyl and carboxyl.

According to another embodiment, the polymerizable molecule(s) are of formula (II) in which Z denotes a radical R₄ and R₃ represents —C(O)—O—R′₄ with R′₄ as defined previously.

According to one embodiment, the polymerizable molecule(s) are of formula (II) in which Z denotes a radical R₄ and R₄ and R′₄, which may be identical or different, represent i) a hydrogen atom, or ii) a (C₁-C₄)alkyl group such as methyl, preferably a hydrogen atom.

According to another embodiment, the polymerizable molecule(s) are of formula (II) in which Z denotes a radical R₄, and R₄ and R′₄, which may be identical or different, represent a (C₁-C₁₆)alkyl group substituted with one or more radicals, which may be identical or different, chosen from the following radicals: amino (NH₂), mono- or di(C₁-C₄)alkylamino such as dimethylamino, hydroxyl (OH), SO₃H, carboxyl (CO₂H), cyano, trifluoromethyl (CF₃), (C₁-C₄)alkyloxycarbonyl, which is preferably monosubstituted; according to this embodiment, R₄ and R′₄, which may be identical or different, in particular represent a (C₁-C₆)alkyl group substituted with one or more radicals, which may be identical or different, chosen from the following radicals: amino (NH₂), mono- or di(C₁-C₄)alkylamino such as dimethylamino, hydroxyl (OH), SO₃H, carboxyl (CO₂H), cyano, said radical preferably being monosubstituted, such as the 2-hydroxypropyl radical.

According to another embodiment, the polymerizable molecule(s) are of formula (II) in which Z denotes a radical R₄, and R₄ and R′₄, which may be identical or different, represent a (C₁-C₁₆)alkyl group interrupted with one or more heteroatoms, such as O, S and N(R₅) with R₅ as defined previously, and preferably R₅ denoting H or Me; according to this embodiment, R₄ and R′₄, which may be identical or different, represent a (C₁-C₁₆)alkyl group interrupted with one or more non-adjacent oxygen atoms and especially a radical —(CH₂CH₂O)ₙ—R in which n denotes an integer equal to 0 or 1 or 2 or 3 or 4 or 5 and R denotes a C₁-C₆ alkyl radical such as methyl or ethyl, or alternatively R₄ and R′₄ independently denote a 2-ethoxyethyl radical.

According to a particular variant of the invention, the polymerizable molecule(s) are of formula (II) in which Z denotes a radical of formula (Z′).

Preferably, the polymerizable molecules of formula (II) are such that Z denotes a radical of formula (Z′) and (Z′) represents a group chosen from:

a) —(CH₂)ₙX with X representing a group from among: hydroxyl, amino, (di)(C1-C4)alkylamino such as dimethylamino, sulfonic —SO₃H, carboxyl or cyano and n representing an integer between 1 and 5 inclusive;

b) —(CH₂CH₂O)ₙ—R and n is as defined above, R representing a hydrogen atom or a (C₁-C₄)alkyl group such as methyl;

c) —(CH₂)ₙ(CF₃)ₘ and n is equal to 1, 2 or 3 and m is an integer between 1 and 8 inclusive.

According to one embodiment, the polymerizable molecule(s) are of formula (II) in which Z denotes a radical of formula (Z′), and Y₁ₐ and Y₂ₐ represent an oxygen atom.

According to one embodiment, the polymerizable molecule(s) are of formula (II) in which Z denotes a radical of formula (Z′) and R₁ represents a hydrogen atom or a (C₁-C₄)alkyl group such as methyl.

According to one embodiment, the polymerizable molecule(s) are of formula (II) in which Z denotes a radical of formula (Z′) and R₁ represents a hydrogen atom.

According to another embodiment, the polymerizable molecule(s) are of formula (II) in which Z denotes a radical of formula (Z′) and R₁ represents a (C₁-C₄)alkyl group substituted with a radical chosen from hydroxyl and —C(Y′₁)—Y′₂—R′₄, in particular —C(O)—O—R′₄ with R′₄ as defined previously; preferably R₁ denotes a (C₁-C₄)alkyl group substituted with a carboxyl group such as carboxymethyl.

According to one embodiment, the polymerizable molecule(s) are of formula (II) in which Z denotes a radical of formula (Z′) and R₂ represents a hydrogen atom or a (C₁-C₄)alkyl group optionally substituted with one or more radicals chosen from hydroxyl, (di)(C₁-C₄)(alkyl)amino, (C₁-C₄)alkoxy and carboxyl radicals; preferably, R₂ represents a hydrogen atom.

According to one embodiment, the polymerizable molecule(s) are of formula (II) in which Z denotes a radical of formula (Z′) and R₃ represents i) a hydrogen atom, ii) a (C₁-C₄)alkyl group optionally substituted with one or more radicals chosen from hydroxyl, (di)(C₁-C₄)(alkyl)amino, (C₁-C₄)alkoxy and carboxyl radicals; iii) —C(O)—O—R′₄.

According to one embodiment, the polymerizable molecule(s) are of formula (II) in which Z denotes a radical of formula (Z′) and R₃ represents i) a hydrogen atom. According to another embodiment, the polymerizable molecule(s) are of formula (II) in which Z denotes a radical of formula (Z′) and R₃ represents a (C₁-C₄)alkyl group optionally substituted with a radical chosen from hydroxyl and carboxyl.

According to another embodiment, the polymerizable molecule(s) are of formula (II) in which Z denotes a radical of formula (Z′) and R₃ represents —C(O)—O—R′₄ with R′₄ as defined previously.

According to one embodiment, the polymerizable molecule(s) are of formula (II) in which Z denotes a radical of formula (Z′), and R₄ and R′₄, which may be identical or different, represent i) a hydrogen atom, or ii) a (C₁-C₄)alkyl group such as methyl, preferably a hydrogen atom.

According to another embodiment, the polymerizable molecule(s) are of formula (II) in which Z denotes a radical of formula (Z′), and R₄ and R′₄, which may be identical or different, represent a (C₁-C₁₆)alkyl group substituted with one or more radicals, which may be identical or different, chosen from the following radicals: amino (NH₂), mono- or di(C₁-C₄)alkylamino such as dimethylamino, hydroxyl (OH), SO₃H, carboxyl (CO₂H), cyano, trifluoromethyl (CF₃), (C₁-C₄)alkyloxycarbonyl, which is preferably monosubstituted; according to this embodiment, R₄ and R′₄, which may be identical or different, in particular represent a (C₁-C₆)alkyl group substituted with one or more radicals, which may be identical or different, chosen from the following radicals: amino (NH₂), mono- or di(C₁-C₄)alkylamino such as dimethylamino, hydroxyl (OH), SO₃H, carboxyl (CO₂H), cyano, said radical preferably being monosubstituted, such as the 2-hydroxypropyl radical.

According to another embodiment, the polymerizable molecule(s) are of formula (II) in which Z denotes a radical of formula (Z′), and R₄ and R′₄, which may be identical or different, represent a (C₁-C₁₆)alkyl group interrupted with one or more heteroatoms, such as O, S and N(R₅) with R₅ as defined previously, and preferably R₅ denoting H or Me; according to this embodiment, R₄ and R′₄, which may be identical or different, represent a (C₁-C₁₆)alkyl group interrupted with one or more non-adjacent oxygen atoms and especially a radical —(CH₂CH₂O)ₙ—R in which n denotes an integer equal to 0 or 1 or 2 or 3 or 4 or 5 and R denotes a C₁-C₆ alkyl radical such as methyl or ethyl, or alternatively R₄ and R′₄ independently denote a 2-ethoxyethyl radical.

According to another embodiment, the polymerizable molecule(s) are of formula (II) in which Z denotes a radical of formula (Z′) and alk denotes a (C₁-C₁₆)alkylene radical (divalent radical) optionally interrupted with one or more oxygen or sulfur atoms or with one or more NH groups, said divalent radical alk preferably denoting a (C₁-C₁₆)alkylene radical (divalent radical) optionally interrupted with one or more oxygen atoms, such as a radical —(CH₂—CH₂—O)_{q} with q=1 to 8.

According to another embodiment, the polymerizable molecule(s) are of formula (II) in which Z denotes a radical of formula (Z′) and alk denotes a (C₁-C₁₆)alkylene and preferably (C₁-C₆)alkylene radical (divalent radical) such as a C₅ alkyl radical or a C₄ alkyl radical or a C₃ alkyl radical, said radical alk being optionally substituted with one or more hydroxyl radical and/or one or more radicals of formula (Z″) as defined previously, preferably optionally substituted with a hydroxyl radical and substituted with one or two radicals of formula (Z″) as defined previously. According to this embodiment, when alk is substituted with one or two radicals of formula (Z″):
when p=1, Y_{2c} preferably denotes an oxygen atom;
ALK preferably denotes a C₁-C₄ radical such as methylene or propylene;
Y_{1b} and Y_{2b} are preferably identical and in particular denote oxygen;
R_{1b}, R_{2b} and R_{3B} are preferably identical and in particular denote hydrogen.

More particularly, the polymerizable molecule(s) are chosen from those of formula (IIa) below:

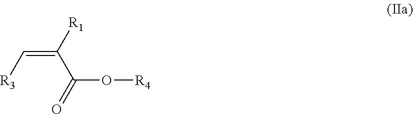

and also the organic or mineral acid or base salts thereof, the optical isomers, geometrical isomers and tautomers thereof, and the solvates thereof such as hydrates; in which formula (IIa) R₁, R₃ and R₄ are as defined previously.

In another particular variant, the polymerizable molecule(s) are chosen from those of formula (IIb) below:

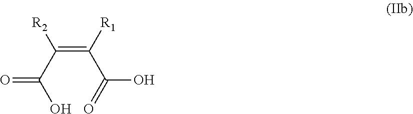

and also the organic or mineral acid or base salts thereof, the optical isomers, geometrical isomers and tautomers thereof, and the solvates thereof such as hydrates; in which formula (IIb) R₁ and R₂ are as defined previously, in particular chosen from hydrogen or (C₁-C₄)alkyl such as methyl, preferably hydrogen.

According to another variant, the polymerizable molecules are chosen from those of formula (IIc) below:

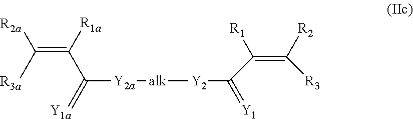

and also the organic or mineral acid or base salts thereof, the optical isomers, geometrical isomers and tautomers thereof, and the solvates thereof such as hydrates;
in which formula (IIc) R₁, R₂, R₃, Y₁, Y₂, alk, Y₂ₐ, Y₁ₐ, R₁ₐ, R₂ₐ and R₃ₐ are as defined previously, in particular such that R₁═R₁ₐ and R₂═R₂ₐ and R₃═R₃ₐ and Y₁═Y₁ₐ and Y₂═Y₂ₐ. In particular, the polymerizable molecules are chosen from those of formula (IIc) for which:
R₁ and R₁ₐ are identical and denote hydrogen or (C₁-C₄)alkyl such as methyl, preferably hydrogen;
Y₂ and Y₂a are identical and denote an oxygen atom or NR₅ as defined previously, and in particular Y₂ and Y₂a are identical and denote oxygen or NH;
Y₁ and Y₁ₐ are identical and denote an oxygen atom;
alk denotes a (C₁-C₁₆)alkylene radical (divalent radical) optionally interrupted with one or more oxygen atoms and/or optionally substituted with one or more radicals chosen from hydroxyl and/or a radical of formula (Z″) as defined previously, said radical(s) (Z″), when they are present, preferably being such that R_{1b} and R_{2b} and R_{3b} denote hydrogen, Y_{1b} and Y_{2b} denote oxygen, ALK denotes a (C₁-C₄)alkylene radical (divalent radical) such as methylene or propylene and p is equal to 0 or 1; Y_{2c}, when it is present, denotes an oxygen atom.

According to this variant, the polymerizable molecules are chosen from compounds (B′) to (d) below:

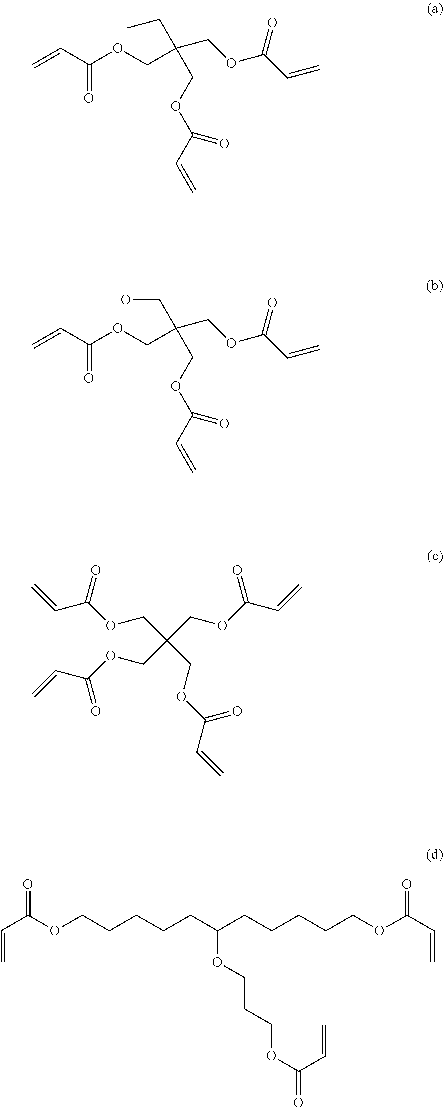

Preferably, the polymerizable molecule(s) are chosen from ((C₁-C₄)alkyl)acrylic acid such as methacrylic acid, acrylic acid and itaconic acid, in particular itaconic acid and also the organic or mineral base salts thereof and the solvates thereof such as hydrates.

According to a particular embodiment, compositions (A), (B) or (B′) comprise one or more polymerizable molecules as defined previously, in an amount inclusively between 0.01% and 50% by weight relative to the total weight of compositions (A), (B) or (B′), in particular between 0.1% and 40%, more particularly between 1% and 30%, preferentially between 2% and 20%, more preferentially between 5% and 10% by weight relative to the total weight of compositions (A), (B) or (B′).

iii) The Amines

Compositions (A), (B) or (B′) optionally comprise one or more amines.

According to one variant, compositions (A), (B) or (B′) comprise one or more amines.

According to the invention, the term “amines” means any organic molecule comprising at least one primary, secondary or tertiary amine group, other than i) the riboflavins of formula (I), and other than ii) the polymerizable molecules. The amines may be aromatic or nonaromatic, saturated or unsaturated, oligomeric or polymeric, and natural or synthetic.

According to a preferred embodiment, the amine(s) of the invention are present in compositions (A), (B) or (B′) in an amount of between 0.01% and 15% by weight relative to the total weight of the composition which comprises same, more particularly between 0.05% and 5%, more preferentially between 0.1% and 3% by weight relative to the total weight of the composition which comprises same.

According to a particular embodiment of the invention, compositions (A), (B) or (B′) of the invention comprise at least one amino acid, preferably a natural amino acid.

Preferably, the amino acid(s) are chosen from the compounds of formula (III) below:

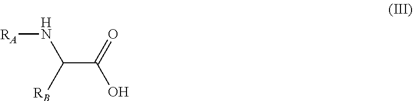

and also the organic or mineral acid or base salts thereof, the optical isomers, geometrical isomers and tautomers thereof, and the solvates thereof such as hydrates;
R_{A} represents a hydrogen atom, a (C₁-C₄)alkyl or (C₁-C₄)alkylcarbonyl group or else forms, together with the radical R_{B} and the nitrogen and carbon atom that bear them, respectively, a 5- to 10-membered monocyclic or bicyclic heterocycle such as pyrrolidinyl; preferably, R_{A} represents a hydrogen atom;
R_{B} represents a hydrogen atom or a (C₁-C₆)alkyl group optionally substituted with one or more groups chosen from i) —Z—C(Z′)—Z″—R_{C} with Z, Z′, and Z″, which may be identical or different, representing an oxygen or sulfur atom, and N(R_{D}), R_{C} and R_{D}, which may be identical or different, representing a hydrogen atom or a (C₁-C₄)alkyl group; preferably, Z, Z′ and Z″ represent a group N(R_{D}) such as NH; ii) (hetero)aryl such as imidazolyl, indolyl, phenyl, optionally substituted especially with a hydroxyl group; iii) (di)(C₁-C₄)(alkyl)amino, iv) —C(Z)—Z″—R_{C}, with Z′, Z″ and R_{C} as defined above, in particular Z′ represents an oxygen atom, Z″ represents an oxygen atom or a group N(R_{D}) such as NH, and R_{C} represents a hydrogen atom; v) —Z′″—R_{C}, with Z′″ representing an oxygen, sulfur or selenium atom or an NH group and R_{C} is as defined previously; in particular, R_{B} represents a (C₁-C₆)alkyl group optionally substituted with i) —Z—C(Z′)—Z″—R_{C}, with Z, Z′, and Z″, which may be identical or different, representing an oxygen or sulfur atom, and N(R_{D}), R_{C} and R_{D}, which may be identical or different, representing a hydrogen atom or a (C₁-C₄)alkyl group; preferably Z, Z′ and Z″ represent a group N(R_{D}) such as NH;
   or R_{A} and R_{B} form, together with the nitrogen atom which bears R_{A} and with the carbon atom which bears R_{B}, a saturated 5- or 6-membered, preferably 5-membered, heterocycle such as a pyrrolidine ring.
   Said amino acids of formula (III) may be of L (levorotatory) or D (dextrorotatory) configuration or in a mixture, preferably of L configuration.

In particular, the amino acid(s) are chosen from alanine, arginine and proline, preferably L-alanine, L-arginine and L-proline, in particular L-arginine or L-proline, more preferentially L-arginine.

According to a preferred embodiment, the amino acid(s) of formula (III) of the invention are present in composition (A) or in composition (B) in an amount of between 0.01% and 15% by weight relative to the total weight of the composition which comprises same, more particularly between 0.05% and 5%, more preferentially between 0.1% and 3% by weight relative to the total weight of the composition which comprises same.

According to another particular embodiment of the invention, composition (A) or (B) of the invention comprises at least one amine comprising an optionally substituted (hetero)aryl group, in particular of formula (IV):

CYC-(ALK)ₚ—NRₑR_{f}

and also the organic or mineral acid or base salts thereof, the optical isomers thereof, and the solvates thereof such as hydrates;
in which formula (IV):
CYC represents an optionally substituted heterocycle, cycloalkyl, aryl or heteroaryl group, preferably aryl such as phenyl,
ALK represents a (C₁-C₆)alkylene group,
Rₑ and R_{f}, which may be identical or different, represent a hydrogen atom or a (C₁-C₆)alkyl group; and
p is equal to 0 or 1.

Preferably, the compounds of formula (IV) are a benzylamine.

According to a preferred embodiment, the amine(s) of formula (IV) are present in composition (A) or in composition (B) in an amount of between 0.01% and 20% by weight relative to the total weight of the composition which comprises same, more particularly between 0.05% and 10%, more preferentially between 0.05% and 5% by weight relative to the total weight of the composition which comprises same.

According to yet another particular embodiment of the invention, composition (A), (B) or (B′) comprises at least one amino monosaccharide or at least one amino polysaccharide.

More preferentially, the monosaccharide(s) bearing amine group(s) a) of the invention are hexosamines of formula (V) and also the salts thereof with an organic or mineral acid such as hydrochloric acid, and also the α or β anomers thereof, the optical isomers thereof of L or D configuration, and the solvates thereof such as hydrates:

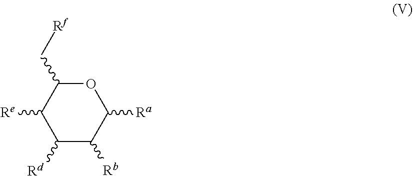

in which formula (V):
R^{a}, R^{b}, R^{d}, R^{e} and R^{f}, which may be identical or different, represent i) a hydroxyl group, ii) a (C₁-C₄)alkoxy group, the alkyl group of which may be optionally substituted, especially with one or more hydroxyl groups, iii) a carboxyl group, and iv) a group NR₁R₂, with R₁ and R₂, which may be identical or different, chosen from a hydrogen atom, (C₁-C₄)alkyl and —C(O)—R′₁ with R′₁ being as defined previously; preferably, R₁ and R₂ represent i) a hydrogen atom or ii) an alkylcarbonyl group —C(O)—R′₁ with R′₁ representing a (C₁-C₄)alkyl group such as methyl;
it being understood that at least one of the radicals R^{a}, R^{b}, R^{d}, R^{e} and R^{f} represents a group NR₁R₂; preferably at least one of the radicals R^{a}, R^{b}, R^{d}, R^{e} and R^{f} represents a group NR₁R₂ and the radicals different from NR₁R₂ denote a hydroxyl group; more particularly, R^{b} represents a group NR₁R₂, preferentially NH₂, and R^{a}, R^{d}, R^{e} and R^{f} represent a hydroxyl group. Preferably, the compounds of formula (V) are of D configuration, also referred to as D-glucopyrans. In particular, the compounds of formula (V) are of β (beta) anomeric configuration. According to a particular mode, the monosaccharide(s) bearing amine group(s) a) of the invention are chosen from the compounds of formula (V) below and also the salts thereof with an organic or mineral acid, preferably a mineral acid such as hydrochloric acid, and also the α or β anomers thereof, the optical isomers thereof of L or D configuration, preferably D configuration, and the solvates thereof such as hydrates:

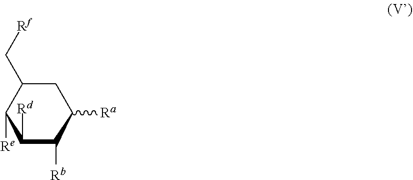

in which formula (V′) R^{a}, R^{b}, R^{d}, R^{e} and R^{f} are as defined for (V) above.

According to a preferred embodiment, the amino monosaccharide(s) of the invention are present in compositions (A), (B) or (B) in an amount of between 0.01% and 20% by weight relative to the total weight of the composition which comprises same, more particularly between 0.05% and 10%, more preferentially between 0.1% and 5% by weight relative to the total weight of the composition which comprises same.

According to yet another particular embodiment of the invention, composition (A), (B) or (B) comprises at least one polysaccharide bearing an amino group, and also the organic or mineral acid salts thereof, the α or β anomers thereof, the optical isomers thereof of L or D configuration and the solvates thereof such as hydrates.

According to a preferred embodiment of the invention, the amino polysaccharide(s) have an average molecular weight MW of less than or equal to 400 kDa, particularly less than 200 kDa.

According to a particular embodiment of the invention, the amino polysaccharide(s) are chosen from those bearing C₅-C₇ saccharide units and also the organic or mineral acid salts thereof, the α or β anomers thereof, the optical isomers thereof of L or D configuration, and the solvates thereof such as hydrates.

According to one particular embodiment, the saccharide units of the amino polysaccharide(s) are linked together between the C1 carbon atoms of one saccharide unit and the C₄ carbon atoms of the other saccharide unit, denoted (1→4), such as the amino polysaccharides of formula (VI) below, and also the organic or mineral acid salts thereof, the α or β anomers thereof, the optical isomers thereof of L or D configuration, and the solvates thereof such as hydrates:

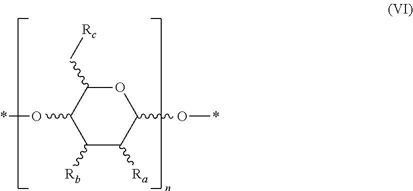

in which formula (VI):
the Rₐ, R_{b}, R_{c} radicals of each saccharide unit may be identical or different;
n is an integer greater than or equal to 2, particularly between 3 and 3000 inclusive, and more particularly between 5 and 2500, preferentially between 10 and 2300;
Rₐ, R_{b}, and R_{c}, which may be identical or different, represent i) a hydroxyl group, ii) a (C₁-C₄)alkoxy group, the alkyl group of which may be optionally substituted, especially with one or more hydroxyl groups, iii) a carboxyl group, and iv) a group NR₁R₂, with R₁ and R₂ as defined above, in particular R₁ and R₂ are chosen from a hydrogen atom and —C(O)—R′₁ in which R′₁ is as defined above; preferably R₁ and R₂ represent i) a hydrogen atom or ii) —C(O)—R′₁ with R′₁ representing a (C₁-C₄)alkyl group such as methyl; it being understood that at least one of the Rₐ, R_{b} or R_{c} radicals of at least one saccharide unit represents a group NR₁R₂ and that at least one of the groups NR₁R₂ of at least one saccharide unit represents an NH₂ group; preferably, Rₐ of at least one saccharide unit represents a group NR₁R₂ with R₁ which represents a hydrogen atom and R₂ is chosen from i) a hydrogen atom or ii) a group —C(O)—R′₁, and R_{b} and R_{c} represent a hydroxyl group, it being understood that at least one of the groups NR₁R₂ of at least one saccharide unit represents an NH₂ group.

More particularly, the amino polysaccharide(s) of the invention are of formula (VI₁) below, and also the organic or mineral acid salts thereof, the α or β anomers thereof, the optical isomers thereof of L or D configuration, and the solvates thereof such as hydrates:

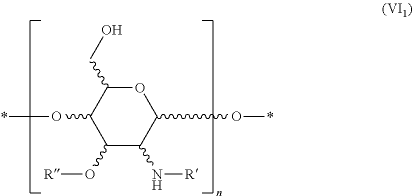

in which formula (VW:
R′ represents a hydrogen atom or a (C₁-C₄)alkylcarbonyl group such as acetyl CH₃—C(O)—;
R″ represents a hydrogen atom or a (C₁-C₄)alkyl group optionally substituted with a carboxyl group such as —CH(CO₂H)—CH₃;
n is an integer greater than or equal to 2, particularly between 3 and 3000 inclusive, more particularly between 5 and 2500, preferentially between 10 and 2300;
   it being understood that in the polysaccharide (VI₁) at least one saccharide unit bears an NH₂ amino group and at least one other saccharide unit bears at least one N(H)—R′ group with R′ representing a (C₁-C₄)alkylcarbonyl group such as acetyl CH₃—C(O)—.

Preferentially, the amino polysaccharide(s) of the invention are chosen from chitin and chitosan and derivatives thereof, preferably chitosan.

More particularly, the amino polysaccharide(s) of the invention are chosen from those of formula (VI₂) below, and also the organic or mineral acid salts thereof, and the solvates thereof such as hydrates:

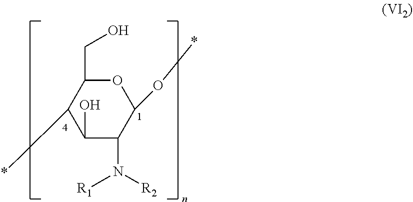

in which formula (VI₂):
R₁ and R₂ are as defined in formula (VI) or (VI₁); and
n is an integer greater than or equal to 2, particularly between 3 and 3000 inclusive, more particularly between 5 and 2500, preferentially between 10 and 2300;
   it being understood that in the polysaccharide of formula (VI₂) at least one saccharide unit bears an amino group NH₂ and at least one other saccharide unit bears a group N(H)—R′ with R′ representing a (C₁-C₄)alkylcarbonyl group such as acetyl CH₃—C(O)—.

More particularly, the amino polysaccharide(s) of the invention are chosen from the chitosans of formula (VI₃) below, and also the organic or mineral acid salts thereof, and the solvates thereof such as hydrates:

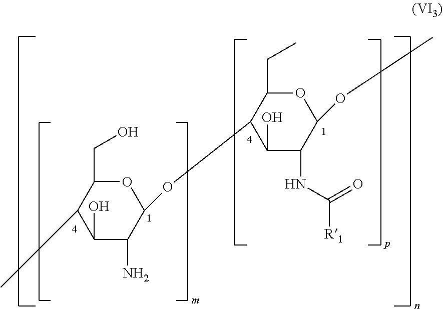

in which formula (VI₃):
R′₁ represents a (C₁-C₄)alkyl group such as methyl; and
n is an integer greater than or equal to 2, particularly between 3 and 3000 inclusive, more particularly between 5 and 2500, preferentially between 10 and 2300;
p is greater than 0 and ranges up to 0.5, preferably from 0.05 to 0.3, and better still from 0.1 to 0.20 such as 0.15 with m+p being equal to 1;
   it being understood that in the chitosan at least one saccharide unit bears an amino group NH₂ and at least one other saccharide unit bears a group N(H)—R′₁ with R′ representing a (C₁-C₄)alkylcarbonyl group such as acetyl CH₃—C(O)—.

For example, when m=0.7, p=0.3 this means that 70% of the amine groups are free (unsubstituted) and 30% of the amino groups are N-alkyl(C₁-C₄)carbonyl groups, in particular N-acetyl groups, corresponding to the chitosan of formula:

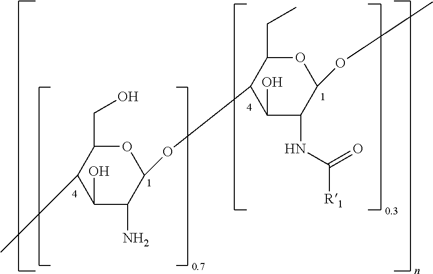

with n as defined above.

According to another form of the invention, the amino polysaccharide(s) of the invention denote a single amino polysaccharide, in particular a chitosan or the organic or mineral acid salts thereof or more particularly the organic acid salts thereof such as the lactic acid salt thereof, the α or β anomers thereof, the optical isomers thereof of L or D configuration, and the solvates thereof such as hydrates.

According to a preferred embodiment, the amino polysaccharide(s) of the invention in particular of formula (VI), (VI₁), (VI₂) or (V₃) as defined previously are present in composition (A) or (B) in an amount of between 0.01% and 20% by weight relative to the total weight of the composition which comprises same, more particularly between 0.05% and 10%, more preferentially between 0.1% and 5% by weight relative to the total weight of the composition which comprises same.

According to a particular embodiment, composition (A), (B) or (B′) comprises at least one amino acid of formula (III) and/or at least one amino a saccharide of formula (V) or (V) and/or at least one amino polysaccharide, these compounds being present in compositions (A), (B) or (B′) in amount(s) of between 0.01% and 20% by weight relative to the total weight of the composition which comprises same, more particularly between 0.05% and 10%, more preferentially between 0.05% and 5% by weight relative to the total weight of the composition which comprises same.

Alcohols

Compositions (A), (B) or (B′) optionally comprise one or more alcohols.

According to another variant, compositions (A), (B) or (B′) comprise one or more alcohols.

According to the invention, the term “alcohol” means any organic molecule comprising at least one hydroxyl group, other than i) the riboflavins of formula (I), and other than ii) the polymerizable molecules. The alcohols may be aromatic or nonaromatic, saturated or unsaturated, oligomeric or polymeric, and natural or synthetic.

According to a particular embodiment of the invention, composition (A), (B) or (B′) of the invention comprises at least one alcohol comprising an optionally substituted (hetero)aryl group, in particular of formula (IV′):

CYC—(X)_{q}-(ALK)ₚ—OH(IV′)

and also the organic or mineral acid or base salts thereof, the optical isomers thereof, and the solvates thereof such as hydrates;
in which formula (IV′):
CYC represents a heterocycle, cycloalkyl, aryl or heteroaryl group which is optionally substituted, especially with one or more (C₁-C₄)alkyl or (di)(C₁-C₄)(alkyl)amino groups, preferably aryl such as phenyl or cycloalkyl such as cyclohexyl,
ALK represents a (C₁-C₆)alkylene group,
X represents an oxygen atom, a sulfur atom or a radical N(R₅) with R₅ as defined previously, and preferably R₅ denoting H or Me: preferably, X represents an oxygen atom;
p and q, which may be identical or different, are equal to 0 or 1, it being understood that when p is equal to 0, then q is equal to 0 and that p is equal to 1 when q is equal to 1.

Preferably, the compounds of formula (IV) are chosen from benzyl alcohol, phenoxyethanol and N,N-dimethylaminobenzyl alcohol.

According to another preferred embodiment of the invention, the alcohols are chosen from terpenes, more preferentially chosen from menthol, isoborneol and neomenthol.

According to another particular embodiment, the alcohols are chosen from linear or branched, saturated or unsaturated (C₁-C₆) alkanols, such as isopropanol.

According to another particular embodiment of the invention, composition (A) or (B) of the invention comprises at least one alcohol comprising an optionally substituted (hetero)aryl group, in particular of formula (IV″):

RₑR_{f}N-(ALK′)ₚ—CYC-(ALK)ₚ—OH

and also the organic or mineral acid or base salts thereof, the optical isomers thereof, and the solvates thereof such as hydrates;
in which formula (IV):
CYC represents an optionally substituted heterocycle, cycloalkyl, aryl or heteroaryl group, preferably aryl such as phenyl,
ALK and ALK′ independently represents a (C₁-C₆)alkylene group,
Rₑ and R_{f}, which may be identical or different, represent a hydrogen atom or a (C₁-C₆)alkyl group such as methyl; and
p and p′ are independently equal to 0 or 1.
Preferably the compounds of formula (IV″) are chosen from an N,N-dimethylaminobenzyl alcohol.

According to a preferred embodiment, the alcohol(s), in particular the alcohol(s) of formula (IV) or (IV), are present in compositions (A), (B) or (B′) in an amount of between 0.01% and 20% by weight relative to the total weight of the composition which comprises same, more particularly between 0.05% and 10%, more preferentially between 0.05% and 5% by weight relative to the total weight of the composition which comprises same.

According to a preferred embodiment of the invention, compositions (A), (B) or (B) comprise one or more amines, in particular amino acids, and one or more alcohols as defined previously, in an amount inclusively between 0.01% and 30% by weight relative to the total weight of compositions (A), (B) or (B′), in particular between 0.05% and 20%, more particularly between 0.1% and 10%, preferentially between 1% and 5% by weight relative to the total weight of compositions (A), (B) or (B′).

### Step 2)—the Step of Supplying Enemy by Liqht Radiation

According to the process of the invention, step 2) comprises the exposure of keratin materials to at least one artificial and/or natural light radiation which may be continuous or non-continuous, before, during or after application of compositions (A), (B) or (B) as defined previously to said materials.

According to the process of the invention, step 2) in particular comprises the exposure of keratin materials to at least one artificial and/or natural light radiation which may be continuous or non-continuous, before, during or after application of compositions (A), (B) or (B) as defined previously to said materials.

For the purposes of the invention, the term “artificial light radiation” means light radiation other than natural daylight (generated by the sun). In other words, natural daylight (generated by the sun) is not artificial light radiation.

The term “natural radiation” means radiation whose sole light source is that of the day generated by the sun.

Preferably, the exposure of the keratin fibers to artificial light radiation is performed at a wavelength of between 360 and 600 nm, preferably between 375 and 550 nm, more preferentially between 400 and 480 nm.

Preferably, the artificial light radiation has an amount of energy per unit area of greater than or equal to 1 J/cm², more preferentially strictly greater than 1 J/cm², even more preferentially between 1.001 and 100 J/cm², better still between 2 and 50 J/cm², particularly preferably between 3 and 10 J/cm².

Preferably, the artificial light radiation is generated using a device chosen from arc lamps such as xenon lamps and mercury lamps, fluorescent lamps, incandescent lamps such as halogen lamps, light-emitting diodes (LED), organic light-emitting diodes (OLED) and lasers.

Mention may be made, for example, of goLITE BLU from the company Philips, the lamp Energylight HF 3319/01 from the company Philips, the lamps Dayvia White and Messa from the company Solvital, the lamp Lumino Plus from the company Lanaform, the lamp Medibeam from the company Medibeam, the lamp M-LED 01 from the company Meimed, the lamp Lifemax Light Pod from the company Lifemax, the lamp Lite-Pad from the company Reicorp, the lamps Omnilux Clear-U and New-U from the company Omnilux, the 1000 W xenon arc lamp from the company Lot-Oriel and the lamp Camag Box 3 (4×8 W) from the company Camag.

Preferably, the duration of exposure of the keratin materials to artificial or natural light radiation is greater than or equal to 5 seconds, more preferentially between 10 seconds and 60 minutes, even more preferentially between 15 seconds and 50 minutes, and better still between 20 seconds and 40 minutes.

According to a first variant of the invention, the invention relates to a process (1) for treating keratin materials, in particular keratin fibers, especially human keratin fibers such as the hair, comprising:
1) a step of applying a composition (A) comprising:
   i) one or more compounds of formula (I) as defined previously; and
   ii) one or more polymerizable molecules as defined previously;
2) a step of supplying energy to said keratin materials, which consists in exposing said materials to at least one artificial or natural light radiation as defined previously,
   steps 1) and 2) possibly being performed simultaneously or separately, preferably simultaneously or else separately 1) and then 2).

According to another variant of the invention, the invention relates to a process (2) for treating keratin materials, in particular keratin fibers, especially human keratin fibers such as the hair, comprising:

1) a step of applying a composition (B) or (B) as defined previously; and
2) a step of supplying energy to said keratin materials, which consists in exposing said materials to at least one artificial or natural light radiation,
steps 1) and 2) possibly being performed simultaneously with the application, or sequentially, preferably simultaneously with the application or else sequentially 1) and then 2).

Preferably, the process of the invention is process (1).

According to a particular embodiment of the invention, process (1) for treating keratin materials of the invention involves at the same time (simultaneously) steps 1) and 2).

According to one variant, at the same time as the application of composition (A) to the keratin materials, said materials are exposed to at least one artificial and/or natural light radiation.

According to another variant of the invention according to process (2), composition (A) is exposed to at least one artificial and/or natural light radiation to give composition (B), said composition (B) then being applied to the keratin materials.

According to a particular embodiment of the invention, when steps 1) and 2) of the process are performed simultaneously, the duration of exposure of the keratin materials to at least one artificial and/or natural light radiation is between 5 seconds and 3 hours, preferably between 1 minute and 1 hour, such as 15 minutes; optionally followed by a step of rinsing said keratin fibers.

According to a preferred embodiment of the invention, when step 2) is performed after step 1), the duration of exposure of the keratin materials to artificial or natural light radiation is between 5 seconds and 3 hours, preferably between 1 minute and 1 hour, such as 15 minutes; optionally, a step of leaving composition (A) on said keratin fibers for a time of between 5 seconds and 3 hours, preferably between 1 minute and 1 hour, such as 15 minutes, is performed between step 1) and step 2); optionally, a step of rinsing said keratin fibers is performed after step 2).

According to another preferred embodiment of the invention, when step 2) is performed after step 1), the duration of exposure of the keratin materials to artificial or natural light radiation is between 5 seconds and 3 hours, preferably between 1 minute and 1 hour, such as 15 minutes; optionally, a step of leaving composition (A), (B) or (B) on said keratin fibers for a time of between 5 seconds and 3 hours, preferably between 1 minute and 1 hour, such as 15 minutes, is performed between step 1) and step 2); a step of rinsing said keratin fibers is performed after step 1).

Compositions (A), (B) or (B) of the invention may be applied to dry or wet keratin materials, preferably to dry or wet hair, preferably to dry hair.

The bath ratio of the applied compositions may range from 0.1 to 10, more particularly from 0.2 to 5 and preferably between 0.5 and 3. The term “bath ratio” means the ratio between the total weight of the applied composition and the total weight of keratin materials to be treated.

In particular, the step of applying composition (A), (B) or (B) to the keratin materials may be followed by a leave-on time. The leave-on time, i.e. the time of contact of composition (A), (B) or (B′) with the keratin materials, is preferably at least 5 minutes, preferably between 10 and 60 minutes and preferably between 15 and 45 minutes.

Rinsing of the hair may optionally be envisaged after applying composition (A), (B) or (B) and optionally the leave-on time.

The hair may then optionally be wrung dry, preferably wrung dry.

Process for Preparing a Composition (B′)

According to a preferred embodiment of the invention, the process for preparing composition (B) comprises step a) of mixing i) one or more flavin derivatives chosen from the compounds of formula (I) as defined previously with ii) one or more polymerizable molecules chosen from those of formula (II), (IIa), (IIb) or (IIc) as defined previously and with iii) one or more amines and/or one or more alcohols other than the riboflavins of formula (I) and other than the polymerizable molecules of formula (II), (IIa), (IIb) or (IIc), preferably one or more amines chosen from amino acids; followed by a step b) of exposing said mixture to at least one artificial and/or natural light radiation, in particular for at least 10 minutes.

More particularly, the process for preparing composition (B′) comprises step a) of mixing i) one or more flavin derivatives chosen from the compounds of formula (I) as defined previously with ii) one or more polymerizable molecules chosen from those of formula (II), (IIa), (IIb) or (IIc) as defined previously and with iii) one or more amino acids preferably of formula (III) as defined previously; followed by a step b) of exposing said mixture to at least one artificial and/or natural light radiation, in particular radiation, for at least 2 minutes, preferably for between 2 minutes and up to 10 hours, more preferentially between 15 minutes and 5 hours, such as 3 hours.

A subject of the invention is also composition (B′) obtained via the process as described previously.

The process of the invention is preferably performed on keratin fibers, especially human keratin fibers, such as the hair.

According to one variant, the process of the invention is performed on keratin fibers, especially human keratin fibers, such as the hair. According to a particular variant of the invention, the process is performed on damaged, bleached or sensitized keratin fibers.

According to a particular form of the invention, the process of the invention is a process for treating keratin materials, in particular keratin fibers, especially human keratin fibers such as the hair, comprising:
1) a step of applying to dry or wet keratin materials one or more compounds of formula (I) as described previously, or a composition (A), (B) or (B′) containing the compounds of formula (I), optionally in the presence of one or more amines and/or of one or more alcohols as defined previously; and then
1i) optionally a step of leaving on the keratin materials, preferably for at least 1 minute, preferably for a time of between 5 and 60 minutes, more preferentially between 10 and 45 minutes; and then
1ii) optionally wringing out, and then
1iii) optionally drying in the open air or with a heating device which delivers heat of less than or equal to 80° C., for example using a hood or a hairdryer, and then
2) a step of supplying energy to said keratin materials, which consists in exposing said materials to at least one artificial or natural light radiation; and then
2i) a rinsing step.

According to another variant, the process is performed on “natural” keratin fibers, i.e. fibers that have not undergone any chemical treatment such as dyeing, bleaching or permanent-waving.

Compositions (A), (B) and (B′)

The Compositions

The composition(s) of the invention are cosmetic, i.e. they contain a physiologically acceptable medium, that is to say a medium that is compatible with human keratin materials such as the skin (of the body, face, around the eyes or the scalp), the hair, the eyelashes, the eyebrows, bodily hair, the nails or the lips.

The physiologically acceptable medium of the composition(s) used in the process according to the invention is advantageously an aqueous medium. It may be constituted, for example, of water or of a mixture of water and of at least one cosmetically acceptable organic solvent. Examples of organic solvents that may be mentioned include C₂-C₄ lower alcohols, such as ethanol and isopropanol; polyols, especially those containing from 2 to 6 carbon atoms, for instance glycerol, propylene glycol, butylene glycol, pentylene glycol, hexylene glycol, dipropylene glycol or diethylene glycol; polyol ethers, for instance 2-butoxyethanol, propylene glycol monomethyl ether and diethylene glycol monomethyl ether or monoethyl ether; and mixtures thereof.

The cosmetic compositions of the invention are preferably water-based and then comprise water at a concentration ranging from 10% to 99.5%, better still from 30% to 95% and even better still from 50% to 95% by weight relative to the total weight of the composition, and even more preferentially from 60% to 90% by weight relative to the total weight of the composition.

The composition used according to the invention may also contain one or more cosmetic additives chosen from nonionic, anionic, cationic and amphoteric surfactants, vitamins and provitamins other than the compounds of formula (I) including panthenol, fillers, dyestuffs, nacreous agents, opacifiers, sequestrants, film-forming polymers, cationic, anionic or neutral polymers, associative polymers, plasticizers, silicones, thickeners, oils, antifoams, moisturizers, emollients, penetrants, fragrances and preserving agents; preferably one or more nonionic, anionic, cationic or amphoteric surfactants, cationic, anionic or neutral polymers, or associative polymers.

Depending on their nature and the purpose of the composition, the usual cosmetic ingredients may be present in usual amounts, which can be readily determined by those skilled in the art and which may be, for each ingredient, between 0.01% and 80% by weight. Those skilled in the art will take care to select the ingredients included in the composition, and also the amounts thereof, so that they do not harm the properties of the compositions of the present invention.

These compositions may be packaged in pump-action bottles or in aerosol containers, so as to apply the composition in vaporized (lacquer) form or in the form of a mousse. Such packaging forms are indicated, for example, when it is desired to obtain a spray or a mousse, for treating the hair. In these cases, the composition preferably comprises at least one propellant.

pH of the Compositions:

The pH of compositions (A), (B) or (B) is preferably between 3 and 10, more particularly in the region of neutrality between 6 and 8, such as 7.

The pH values may be adjusted with an organic or mineral acid, or with an alkaline agent chosen from mineral or organic or hybrid alkaline agents or mixtures thereof.

The term “organic acid” means an acid, i.e. a compound that is capable of releasing a cation or proton H⁺ or H₃O⁺ in aqueous medium, which comprises at least one optionally unsaturated, linear or branched C₁-C₂₀ hydrocarbon-based chain, a (hetero)cycloalkyl or (hetero)aryl group and at least one acidic chemical function chosen in particular from carboxyl C(O)OH, sulfonic SO₃H, sulfinic SO₂H, phosphonic PO₃H₂ and phosphinic PO₂H₂.

More particularly, the acids used are chosen from hydrochloric acid HCl, hydrobromic acid HBr, sulfuric acid H₂SO₄, alkylsulfonic acids: (C₁-C₆)Alk-S(O)₂OH such as methylsulfonic acid and ethylsulfonic acid; arylsulfonic acids: Ar—S(O)₂OH such as benzenesulfonic acid and toluenesulfonic acid; (C₁-C₆)alkoxysulfinic acids: Alk-O—S(O)OH such as methoxysulfinic acid and ethoxysulfinic acid; aryloxysulfinic acids such as tolueneoxysulfinic acid and phenoxysulfinic acid; phosphoric acid H₃PO₄; triflic acid CF₃SO₃H and tetrafluoroboric acid HBF₄, and carboxylic acid(s) of formula (D) below:

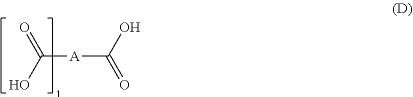

in which formula (D) or a salt thereof:
A represents a saturated or unsaturated, cyclic or non-cyclic, and aromatic or non-aromatic hydrocarbon-based group, which is monovalent when t is 0 or polyvalent when t is greater than or equal to 1, comprising from 1 to 50 carbon atoms, which is optionally interrupted with one or more heteroatoms and/or optionally substituted, especially with one or more hydroxyl groups; preferably, A represents a monovalent (C₁-C₆)alkyl group or a polyvalent (C₁-C₆)alkylene group optionally substituted with one or more hydroxyl groups.

Particularly, the carboxylic acid(s) of formula (D) as defined previously, and preferably the acid(s) used, are an alpha-hydroxy acid such as lactic acids, glycolic acids, tartaric acids or citric acids.

The mineral alkaline agent(s) are preferably chosen from aqueous ammonia, alkaline carbonates or bicarbonates such as sodium or potassium carbonates and sodium or potassium bicarbonates, sodium hydroxide or potassium hydroxide, or mixtures thereof.

According to an advantageous embodiment of the invention, the alkaline agent(s) are organic amines, i.e. they contain at least one substituted or unsubstituted amino group.

The organic alkaline agent(s) are more preferentially chosen from organic amines with a pK_{b} at 25° C. of less than 12, preferably of less than 10 and even more advantageously of less than 6. It should be noted that it is the pK_{b} corresponding to the function of highest basicity.

Hybrid compounds that may be mentioned include the salts of the amines mentioned previously with acids such as carbonic acid or hydrochloric acid. The organic alkaline agent(s) are chosen, for example, from alkanolamines, oxyethylenated and/or oxypropylenated ethylenediamines, amino acids and the compounds of formula (E) below:

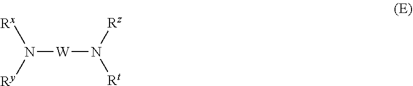

in which formula (E):
W is a divalent C₁-C₆ alkylene radical optionally substituted with a hydroxyl group or a C₁-C₆ alkyl radical, and/or optionally interrupted with one or more heteroatoms such as oxygen or NR^{u};
R^{x}, R^{y}, R^{z}, R^{t} and R^{u}, which may be identical or different, represent a hydrogen atom or a C₁-C₆ alkyl, C₁-C₆ hydroxyalkyl or C₁-C₆ aminoalkyl radical.

Preferably, the alkanolamine is ethanolamine (or monoethanolamine).

In one variant of the invention, the composition comprises, as alkaline agent, one or more alkanolamines (preferably ethanolamine) and aqueous ammonia. In this variant, the alkanolamine(s) are present in a predominant amount relative to the aqueous ammonia.

The examples that follow serve to illustrate the invention without, however, being limiting in nature.

### EXAMPLES

The process according to the invention makes it possible to obtain polymeric compounds which may be generated in keratin fibers or else externally to keratin fibers and then applied to said fibers.

### Example 1

Preparation of a Composition (B) or (B′) Intended to be Applied to Keratin Fibers:

An aqueous composition constituted of sodium riboflavin 5′-monophosphate hydrate (3 g %), L-arginine (3 g %), itaconic acid (38 g %) and water (pH adjusted to 7 with NaOH) was irradiated with a 1000 W xenon arc lamp for 3 hours to create a polymer composition (B), which was then applied to the fiber.

### Example 2

The following compositions were prepared according to the following table:

| | g % ingredients | | | | |
|---|---|---|---|---|---|
| | Sodium salt of | | | | |
| | riboflavin 5′- | | | | |
| | monophosphate | | Itaconic | | |
| Locks* | hydrate | L-arginine | acid | | Irradiation |
| 1 | — | — | — | — | NO |
| (comparative) | | | | | |
| 2 | — | — | — | — | YES |
| (comparative) | | | | | |
| 3 | 3 | — | — | — | NO |
| (comparative) | | | | | |
| 4 | — | 3 | — | — | NO |
| (comparative) | | | | | |
| 5 | — | — | — | 40 | NO |
| (comparative) | | | | | |
| 6 | 3 | — | — | — | YES |
| (comparative) | | | | | |
| 7 | — | 3 | — | — | YES |
| (comparative) | | | | | |
| 8 | — | — | — | 40 | YES |
| (comparative) | | | | | |
| 9 | 3 | 3 | — | — | NO |
| (comparative) | | | | | |
| 10 | 3 | — | — | 40 | NO |
| (comparative) | | | | | |
| 11 | — | 3 | — | 40 | NO |
| (comparative) | | | | | |
| 12 | 3 | 3 | — | — | YES |
| (comparative) | | | | | |
| 13 | — | 3 | — | 40 | YES |
| (comparatif) | | | | | |
| 14 | 3 | — | — | 40 | YES |
| (invention) | | | | | |
| 15 | 3 | 3 | — | 40 | NO |
| (comparative) | | | | | |
| 16 | 3 | 3 | — | 40 | YES |
| (invention) | | | | | |
| 17 | — | — | 1 | — | NO |
| (comparative) | | | | | |
| 18 | — | — | 1 | — | YES |
| (comparative) | | | | | |
| 19 | 0.1 | 0.1 | 1 | — | YES |
| (invention) | | | | | |

*The locks numbered 1 to 19 are made using locks of damaged hair (bleached hair). The word “NO” implies that the processes were performed without natural daylight and without artificial light.

### Preparation of the Samples

2.7 g of locks of 20 cm SA20 bleached hair, which were washed and dried beforehand, were used. 1.5 g of each formula were applied for each gram of dry hair. The samples were left on for 15 minutes, wrapped in a thin transparent plastic film. The film was removed and the hair was irradiated using a 1000 W xenon arc lamp for 15 minutes, and then rinsed with excess water at 37° C., combed and dried under a hood at 60° C. for 10 minutes. The placebo samples were prepared in an identical manner, except that the sample was covered with aluminum foil instead of transparent film (to protect it from irradiation). After drying, the samples were combed (comb passed through five times) and then left suspended vertically.

### Test Method

Effect of a Ponytail

The more the hair is damaged, the more the folding of the sample remains marked on the hair after the stress has been removed.

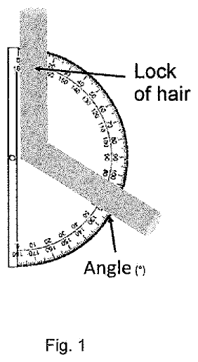

The locks were folded in two (the root toward the end of the hair) and fixed using an elastic band so as to make a loop of about 3 cm, i.e. the length of the loop is 3 cm or, in other words, the elastic band is 3 cm from the bottom of the loop. The looped locks were suspended with the loop at the bottom in a glove box at 80% RH and 26° C. for 2 hours. The locks were removed from the glove box, the elastic band was removed and the locks were suspended vertically. The fold angle was measured according to the reporter's principle (see FIG. 1) immediately after removing the elastic band (initial angle) and then at the end of the study (final angle after 14 hours).

Results

Angle reduction=final angle−initial angle

The greater the angle reduction, the more the fold imprint disappears and the more longitudinal the lock.

| | Initial angle (°) | Final angle (°) | Angle reduction |
|---|---|---|---|
| 1 | 130 | 140 | 10 |
| (comparative) | | | |
| 2 | 140 | 150 | 10 |
| (comparative) | | | |
| 3 | 130 | 130 | 0 |
| (comparative) | | | |
| 4 | 130 | 140 | 10 |
| (comparative) | | | |
| 5 | 130 | 140 | 10 |
| (comparative) | | | |
| 6 | 130 | 140 | 10 |
| (comparative) | | | |
| 7 | 130 | 130 | 0 |
| (comparative) | | | |
| 8 | 120 | 130 | 10 |
| (comparative) | | | |
| 9 | 130 | 140 | 10 |
| (comparative) | | | |
| 10 | 130 | 140 | 10 |
| (comparative) | | | |
| 11 | 130 | 150 | 20 |
| (comparative) | | | |
| 12 | 120 | 130 | 10 |
| (comparative) | | | |
| 13 | 130 | 140 | 10 |
| (comparative) | | | |
| 14 | 140 | 170 | 30 |
| (invention) | | | |
| 15 | 130 | 140 | 10 |
| (comparative) | | | |
| 16 | 140 | 180* | 40* |
| (invention) | | | |
| 17 | 130 | 140 | 10 |
| (comparative) | | | |
| 18 | 130 | 140 | 10 |
| (comparative) | | | |
| 19 | 140 | 170 | 30 |
| (invention) | | | |

*Maximum relaxation (no fold present)

After removing the elastic band, the locks numbered 14, 16 and 19 (present invention) show a better return longitudinal shape, i.e. the fold imprint is no longer visible on these locks. This is not the case for the other comparative locks (1 to 13 and 15, 17 and 18). In addition, it is seen that the damaged locks treated via the process of the invention are perceived as being identical to a natural lock, indicating that the process of the invention has repaired the damaged hair.

### Example 3

Comparative Study (Vs. WO2016/126121)
The use of the same yeast (Saccharomyces cerevisiae) as described in WO2016/126121 is not possible since the details of the supplier are not given neither any procedure to prepare the yeast nor the extraction procedure of the riboflavin. After a literature search it was concluded that the concentration of riboflavin in S. cerevisiae is between 0.8-1.5 μM. Thus the higher concentration of 1.5 μM has been used to do the comparative study. Therefore, a composition comprising 20% by weight of S. cerevisiae comprises an equivalent quantity of riboflavin of 1.13 g×10⁻⁴% by weight. A particular reference for the cytosolic concentration of riboflavin in S. cerevisiae is Biosynthesis of Vitamin B2 (Riboflavin), Annual Review of Nutrition, Vol. 20:153-167 (Volume publication date July 2000)-https://doi.org/10.1146/annurev.nutr.20.1.153
In this study, pentaerythritol tetraacrylate has been used as a polymerizable molecule:

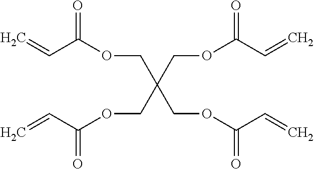 [SMILES: C=CC(=O)OCC(COC(=O)C=C)(COC(=O)C=C)COC(=O)C=C]

The following formulations were prepared. The ingredients were mixed, stirred for one hour in an closed amber vial before use.

| | | | | | | Qsp |
|---|---|---|---|---|---|---|
| | | | | | | 5% |
| | | Penta- | | | | Triton |
| Formu- | | erythritol | | | | X-100⁵ |
| lation | | tetra- | | Benzo- | | in |
| Number | Riboflavin¹ | acrylate² | Urea³ | phenone⁴ | EtOH | Water |
| 1 | 1 g | 5 g | — | — | 50 g | 100 g |
| 2 | 1 g | — | 0.5 g | 0.5 g | 50 g | 100 g |
| 3 | 1.13 × 10⁻⁴ g | — | 0.5 g | 0.5 g | 50 g | 100 g |

¹Sigma-Aldrich - Ref. R7649
²Sigma-Aldrich - Ref. 408263
³Sigma-Aldrich - Ref. U4884
⁴BASF- Ref. Uvinul 40
⁵Sigma-Aldrich - Ref. X-100

### Preparation of Hair Swatches

| | Formulation | |
|---|---|---|
| Example | number | Irradiation |
| 1 (Invention) | 1 | Natural light |
| 2 (Invention) | 1 | Artificial light |
| 3 (Comparative) | 2 | Natural light |
| 4 (Comparative) | 2 | Artificial light |
| 5 (Comparative) | 3 | Natural light |
| 6 (Comparative) | 3 | Artificial light |

The hair swatches (2.7 g, 27 cm length) were placed on a piece of aluminium foil. 5.4 g of each formulation was applied onto each hair swatch. The swatches were covered with a transparent plastic film to reduce evaporation. Examples 1, 3, and 5 were exposed to natural light for 60 minutes. After 30 of the 60 minutes the swatches were turned over to ensure each side of the swatches was exposed to at least 30 minutes of natural light. The irradiance of natural light was measured at 64 microW/cm² using a Thor PM100D Optical Power Meter.
Examples 2, 4 and 6 were irradiated with a home-made array of LED lights emitting light at 450 nm for 60 minutes. The array of LEDs were positioned 5 cm above the swatch and the irradiance measured at 26 mW/cm²). After 30 of the 60 minutes the swatches were turned over to ensure each side of the swatches was irradiated to at least 30 minutes.
After exposure to the light all the swatches were suspended vertically, covered with a towel and left to dry.

### Shampooing

The swatches were subjected to 5 repeat washes. Washing consisted of placing the swatch in a bath containing 100 mL of 3% sodium lauryl ether sulfate (SLES) for 1 minute then rinsing for 10 seconds under tap water. The process is repeated 4 times using fresh SLES solution each time. At the end of the fifth wash and rinse the swatches were dried in a professional helmet hairdryer at 60° C.

### Hair Repair Test—Repeat Combing (Anti Breakage)

The swatches were suspended vertically and combed 30 times with a fine toothed comb at a speed of 30 cm/sec. The amount of broken hair was collected and weighed. The lower the weight of hair collected the better the formulation is at reducing the breakage of hair i.e. repairing the hair.

### Results

| | Amount of |
|---|---|
| Example | broken hair (g) |
| 1 (Invention) | 0.0813 |
| 2 (Invention) | 0.0532 |
| 3 (Comparative) | 0.1336 |
| 4 (Comparative) | 0.1174 |
| 5 (Comparative) | 0.1683 |
| 6 (Comparative) | 0.1749 |

The results show that hair treated according to Examples 1 and 2 with 1% riboflavin and pentaerythritol tetraacrylate have been repaired since there is less hair breakage during the repeated combing. Example 2 using the LED array gave better results than natural light.

1-20. (canceled)

21. A method for treating keratin materials, wherein the method comprises:
1) applying to the keratin materials a composition (A) or a composition (B), wherein the composition (A) comprises i) at least one flavin derivative and ii) at least one polymerizable molecule, and the composition (B) is obtained by exposing the composition (A) to at least one artificial or natural light radiation; and
2) supplying energy to the keratin materials by exposing the keratin materials to at least one artificial or natural light radiation;
wherein the at least one flavin derivative is chosen from compounds of formula (I) below: 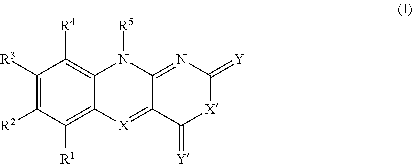
organic or mineral acid or base salts thereof, optical isomers thereof, geometrical isomers thereof, tautomers thereof, or solvates thereof;
wherein in formula (I):
R¹, R², R³ and R⁴, which may be identical or different, are chosen from a hydrogen atom, a halogen atom, or a group chosen from hydroxyl, (C₁-C₆)alkyl, (C₁-C₆)alkoxy, (C₁-C₆)alkylthio, (di)(C₁-C₆)(alkyl)amino, or nitro(so);
R⁵ is chosen from a hydrogen atom or a (C₁-C₈)alkyl group optionally substituted with a group chosen from i) hydroxyl, ii) R⁶—C(Y″)—O—, wherein R⁶ is chosen from a hydrogen atom, a (C₁-C₄)alkyl group, or an aryl(C₁-C₄)alkyl group, and Y″ is chosen from an oxygen atom, a sulfur atom, or N(R⁷), wherein R⁷ is chosen from a hydrogen atom or a (C₁-C₄)alkyl group, iii) phosphoric (HO)₂P(O)—O—, iv) —O—[(HO)P(O)—O]ₙ-Suc-Het, wherein Suc is chosen from a divalent sugar group, and Het is chosen from a heteroaryl group, and n is an integer equal to 0, 1 or 2;
X is chosen from a nitrogen atom or a methylene group C(R⁸), wherein R⁸ is chosen from a hydrogen atom or a (C1-C4)alkyl group;
X′ is chosen from an oxygen atom or a group NR⁹, wherein R⁹ is chosen from a hydrogen atom or a (C1-C6)alkyl group; and
Y and Y′, which may be identical or different, are chosen from an oxygen atom, a sulfur atom, or a group NR¹⁰, wherein R¹⁰ is chosen from a hydrogen atom or a (C1-C6)alkyl group;
wherein steps 1) and 2) are performed simultaneously or step 2) is performed subsequently after step 1); and
wherein the at least one polymerizable molecule comprises at least one unsaturated group.

22. The method of claim 21, wherein in formula (I), R¹ and R⁴ are chosen from a hydrogen atom and R² and R³ are chosen from a (C₁-C₄)alkyl group.

23. The method of claim 21, wherein in formula (I), R⁵ is chosen from a hydrogen atom or a (C₁-C₆)alkyl group.

24. The method of claim 21, wherein in formula (I), R⁵ is chosen from a (C₁-C₆)alkyl group substituted with at least one group, which may be identical or different, chosen from i) hydroxyl, ii) R⁶—C(O)—O—, with R⁶ being chosen from a hydrogen atom, or a (C₁-C₄)alkyl group, iii) phosphoric (HO)₂P(O)—O—, iv) —O—[(HO)P(O)—O]ₙ-Suc-Het, wherein Suc is chosen from a monosaccharide and Het is chosen from an optionally substituted heteroaryl group, and n is an integer equal to 1 or 2.

25. The method of claim 21, wherein in formula (I), taken together or separately, X is a nitrogen atom; X′ is chosen from a group NR⁹ with R⁹ being chosen from a hydrogen atom or a (C₁-C₆)alkyl group; and Y and Y′ are identical and chosen from an oxygen atom or a sulfur atom.

26. The method of claim 21, wherein at least one compound of the compounds of formula (I) is chosen from compounds 1 to 7 below:
(2R,3S,4S)-5-(7,8-dimethyl-2,4-dioxo-3,4 dihydrobenzo[g]pteridin-10(2H)-yl)-2,3,4-trihydroxypentyl dihydrogen phosphate or riboflavin-5′-phosphate (compound 1);
7,8-dimethyl-10-((2R,3R,4S)-2,3,4,5-tetrahydroxypentyl)benzo[g]pteridine-2,4-(3H,10H)-dione, or vitamin B2, lactoflavin (compound 2);
Flavin adenine dinucleotide (FAD) (compound 3);
7,8-dimethylbenzo[g]pteridine-2,4(3H,10H)-dione (compound 4);
7,8,10-trimethylbenzo[g]pteridine-2,4(3H,10H)-dione (compound 5);
Riboflavin tetrabutyrate (compound 6); or
10-methylbenzo[g]pteridine-2,4(3H,10H)-dione (compound 7);

| Structure | |
|---|---|
| 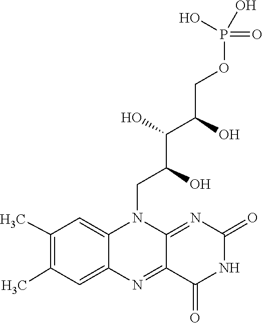 [SMILES: Cc1cc2nc3c(=O)[nH]c(=O)nc-3n(C[C@H](O)[C@H](O)[C@H](O)COP(=O)(O)O)c2cc1C] | 1 |
| 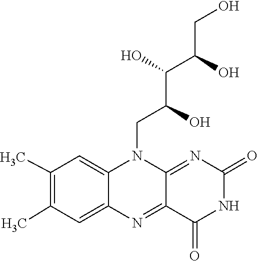 [SMILES: Cc1cc2nc3c(=O)[nH]c(=O)nc-3n(C[C@H](O)[C@H](O)[C@H](O)CO)c2cc1C] | 2 |
| 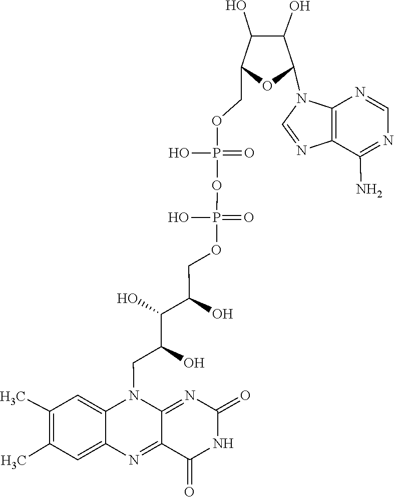 | 3 |
| 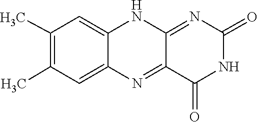 [SMILES: Cc1cc2nc3c(=O)[nH]c(=O)nc-3[nH]c2cc1C] | 4 |
| 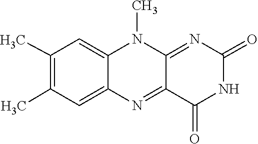 [SMILES: Cc1cc2nc3c(=O)[nH]c(=O)nc-3n(C)c2cc1C] | 5 |
| 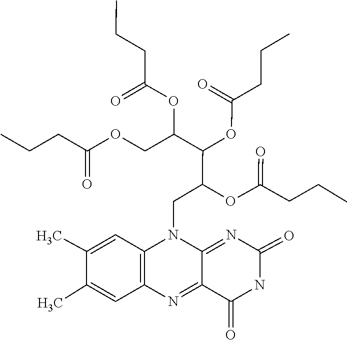 | 6 |
| 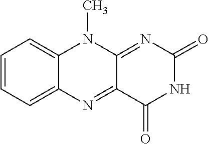 [SMILES: Cn1c2nc(=O)[nH]c(=O)c-2nc2ccccc21] | 7 |

organic or mineral acid or base salts thereof, optical isomers thereof, tautomers thereof, or solvates thereof.

27. The method of claim 21, wherein the at least one polymerizable molecule has a molecular weight of less than or equal to 500 g/mol.

28. The method of claim 21, wherein the at least one polymerizable molecule comprises a photopolymerizable molecule.

29. The method of claim 21, wherein the at least one polymerizable molecule is chosen from compounds of formula (II) below: 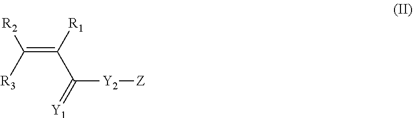
organic or mineral acid or base salts thereof, optical isomers thereof, geometrical isomers thereof, tautomers thereof, or solvates thereof;
wherein in formula (II):
R₁ and R₂, which may be identical or different, are chosen from a hydrogen atom, a (C₁-C₈)alkyl group, or a (C₂-C₈)alkenyl group, wherein the (C₁-C₈)alkyl and the (C₂-C₈)alkenyl groups are:
optionally interrupted with at least one heteroatom chosen from O, S, or N(R₅), wherein R₅ is chosen from a hydrogen atom or a (C₁-C₄)alkyl group, and/or
optionally substituted with at least one radical chosen from the following radicals: hydroxyl, (di)(C₁-C₄)(alkyl)amino, (C₁-C₄)alkoxy, or C(Y′₁)—Y′₂—R′₄;
R₃ is chosen from a hydrogen atom, a (C₁-C₈)alkyl group, a (C₂-C₈)alkenyl group, or a group of —C(Y′₁)—Y′₂—R′₄;
Y₁, Y₂, Y′₁ and Y′₂, which may be identical or different, are chosen from an oxygen atom, a sulfur atom, or N(R₅), wherein R₅ is chosen from a hydrogen atom or a (C₁-C₄)alkyl group; and
R₄ and R′₄, which may be identical or different, are chosen from a hydrogen atom or a (C₁-C₁₆)alkyl group optionally substituted with at least one radical, which may be identical or different, chosen from the following radicals: a) amino (NH₂), or mono- or di(C₁-C₄)alkylamino, b) hydroxyl (OH), c) SO₃H, d) carboxyl (CO₂H), e) cyano, f) (poly)(halo)(C₁-C₄)alkyl, g) (C₁-C₄)alkoxycarbonyl; wherein the alkyl radical is optionally interrupted with at least one heteroatom;
Z is a radical R₄ or a radical (Z′) of formula: 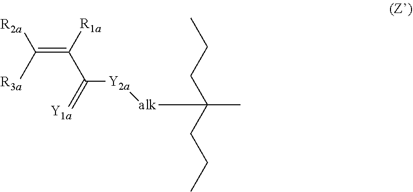
wherein in formula (Z′):
Y₁ₐ, Y₂ₐ, R₁ₐ, R₂ₐ and R₃ₐ have, respectively and independently, the same meanings as that of Y₁, Y₂, R₁, R₂ and R₃;
alk is a divalent (C₁-C₁₆)alkylene radical optionally interrupted with an oxygen atom or a sulfur atom, or with an NH group, the divalent (C₁-C₁₆)alkylene radical being optionally substituted with at least one radical chosen from hydroxyl or a radical of formula (Z″): 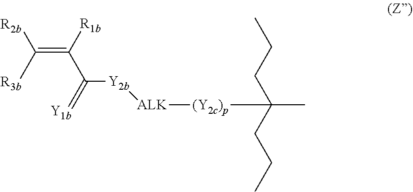
wherein in formula (Z″):
Y_{1b}, Y_{2b}, Y_{2c}, R_{1b}, R_{2b} and R_{3b} have, respectively and independently, the same meanings as that of Y₁, Y₂, R₁, R₂ and R₃; ALK is a (C₁-C₆)alkylene radical (divalent radical); and p is equal to 0 or 1; 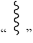
in formula (Z′), the symbol represents the point of attachment between alk and Y₂; and 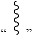
in formula (Z″), the symbol represents the point of attachment between (Y₂)p and ALK.

30. The method of claim 29, wherein R₁ is chosen from a hydrogen atom, a (C₁-C₄)alkyl group, or a (C₁-C₄)alkyl group substituted with a radical chosen from hydroxyl or a —C(Y′₁)—Y′₂—R′₄ group.

31. The method of claim 21, wherein the at least one polymerizable molecule is chosen from compounds of formula (IIa) below: 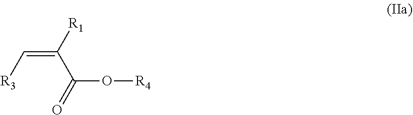
organic or mineral acid or base salts thereof, optical isomers thereof, geometrical isomers thereof, tautomers thereof, or the solvates thereof;
wherein in formula (IIa):
R₁ is chosen from a hydrogen atom, a (C₁-C₈)alkyl group, or a (C₂-C₈)alkenyl group, wherein the (C₁-C₈)alkyl and the (C₂-C₈)alkenyl groups are:
optionally interrupted with a heteroatom chosen from O, S, or N(R₅), wherein R₅ is chosen from a hydrogen atom or a (C₁-C₄)alkyl group; and/or
optionally substituted with a radical chosen from the following radicals: hydroxyl, (di)(C₁-C₄)(alkyl)amino, (C₁-C₄)alkoxy, or —C(Y′₁)—Y′₂—R′₄;
R₃ is chosen from a hydrogen atom, a (C₁-C₈)alkyl group, a (C₂-C₈)alkenyl group, or a —C(Y′₁)—Y′₂-1=1′4 group;
Y′₁ and Y′₂, which may be identical or different, are chosen from an oxygen atom, a sulfur atom, or N(R₅), wherein R₅ is chosen from a hydrogen atom or a (C₁-C₄)alkyl group; and
R₄ and R′₄, which may be identical or different, are chosen from a hydrogen atom or a (C₁-C₁₆)alkyl group optionally substituted with at least one radical, which may be identical or different, chosen from the following radicals: a) amino (NH₂), or mono- or di(C₁-C₄)alkylamino, b) hydroxyl (OH), c) SO₃H, d) carboxyl (CO₂H), e) cyano, f) (poly)(halo)(C₁-C₄)alkyl, or g) (C₁-C₄)alkoxycarbonyl; wherein the alkyl radical is optionally interrupted with at least one heteroatom.

32. The method of claim 21, wherein the at least one polymerizable molecule is chosen from compounds of formula (IIb) below: 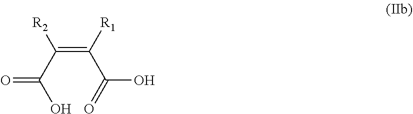
organic or mineral acid or base salts thereof, optical isomers thereof, geometrical isomers thereof, tautomers thereof, or solvates thereof;
wherein in formula (IIb), R₁ and R₂, which may be identical or different, are chosen from hydrogen or (C₁-C₄)alkyl.

33. The method of claim 21, wherein the at least one polymerizable molecule is chosen from compounds of formula (IIc) below: 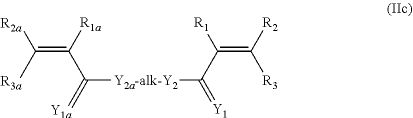
organic or mineral acid or base salts thereof, optical isomers thereof, geometrical isomers thereof, tautomers thereof, or solvates thereof;
wherein in formula (IIc), R₁, R₂, R₃, Y₁, Y₂, alk, Y₂ₐ, Y₁ₐ, R₁ₐ, R₂ₐ, and R₃ₐ are as defined for formula (II), such that R₁═R₁ₐ, R₂═R₂ₐ, R₃═R₃ₐ, Y₁═Y₁ₐ, and Y₂═Y₂ₐ, wherein:
R₁ and R₂ are chosen from a hydrogen atom, a (C₁-C₈)alkyl group, or a (C₂-C₈)alkenyl group, wherein the (C₁-C₈)alkyl group or the (C₂-C₈)alkenyl group is:
optionally interrupted with at least one heteroatom chosen from O, S, or N(R₅), wherein R₅ is chosen from a hydrogen atom or a (C₁-C₄)alkyl group, and/or
optionally substituted with at least one radical chosen from the following radicals: hydroxyl, (di)(C₁-C₄)(alkyl)amino, (C₁-C₄)alkoxy or —C(Y′₁)—Y′₂—R′₄,
R₃ and Ria are chosen from a hydrogen atom, a (C₁-C₈)alkyl group, a (C₂-C₈)alkenyl group, or a —C(Y′₁)—Y′₂—R′₄ group;
Y₁, Y₂, Y′₁ and Y′₂ are chosen from an oxygen atom, a sulfur atom, or N(R₅), wherein R₅ is chosen from a hydrogen atom or a (C₁-C₄)alkyl group; and
alk is a divalent (C₁-C₁₆)alkylene radical optionally interrupted with at least one oxygen atom, a sulfur atom, or an NH group, wherein the divalent (C₁-C₁₆)alkylene radical is optionally substituted with at least one radical chosen from hydroxyl or a radical of formula (Z″): and 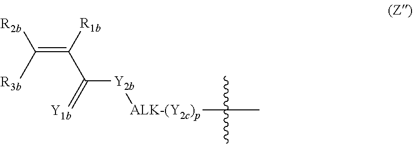
wherein in formula (Z″):
Y_{1b}, Y_{2b}, Y_{2c}, R_{1b}, R_{2b} and R_{3b} have, respectively and independently, the same meanings as that of Y₁, Y₂, R₁, R₂ and R₃; ALK is a (C₁-C₆)alkylene radical (divalent radical); and p is equal to 0 or 1.

34. The method of claim 21, wherein the at least one polymerizable molecule is chosen from compounds (a) to (d) below:

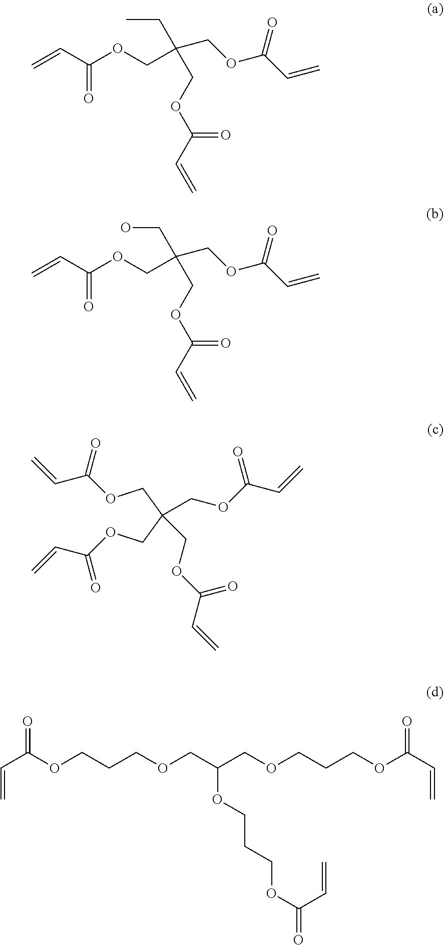

35. The method of claim 21, wherein:
the at least one compound of formula (I) is present in an amount ranging from 0.01% to 30% by weight, relative to the total weight of composition (A) or the composition (B), and/or
the at least one polymerizable molecule is present in an amount ranging from 0.01% to 50% by weight, relative to a total weight of the composition (A) or the composition (B).

36. The method of claim 21, wherein the composition (A) or the composition (B) comprises at least one amine other than i) the flavin of formula (I), and other than ii) the at least one polymerizable molecule; wherein the at least one amine is chosen from aromatic, non-aromatic, saturated, unsaturated, oligomeric, polymeric, natural, or synthetic amines.

37. The method of claim 36, wherein the at least one amine is an amino acid chosen from compounds of formula (III) below: 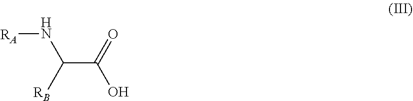
organic or mineral acid or base salts thereof, optical isomers thereof, geometrical isomers thereof, tautomers thereof, or solvates thereof;
wherein in formula (III):
R_{A} is chosen from a hydrogen atom, a (C₁-C₄)alkyl group, or (C₁-C₄)alkylcarbonyl group, forming, together with the radical R_{B}, the nitrogen, and carbon atom that bear R_{A}, R_{B} and the nitrogen, respectively, a 5- to 10-membered monocyclic or bicyclic heterocycle; and
R_{B} is chosen from a hydrogen atom or a (C₁-C₆)alkyl group optionally substituted with at least one group chosen from i) —Z—C(Z′)—Z″—R_{C}, wherein Z, Z′, or Z″, which may be identical or different, are chosen from an oxygen atom, a sulfur atom, or N(R_{D}), wherein R_{C} and R_{D}, which may be identical or different, are chosen from a hydrogen atom or a (C₁-C₄)alkyl group; ii) (hetero)aryl; iii) (di)(C₁-C₄)(alkyl)amino; iv) —C(Z)—Z″—R_{D}, wherein Z′ is chosen from an oxygen atom, Z″ is chosen from an oxygen atom or a group N(R_{D}), and R_{c} is chosen from a hydrogen atom; v) —Z″—R_{C}, with Z′″ being chosen from an a hydrogen atom or a (C₁-C₄)alkyl group oxygen, sulfur or selenium atom, or an NH group, and with R_{C} being chosen from a hydrogen atom or a (C₁-C₄)alkyl group.

38. The method of claim 21, wherein the step of exposing the keratin materials to at least one artificial or natural light radiation comprises exposing the keratin materials to an artificial light radiation with a wavelength ranging from 360 nm to 600 nm; and wherein the artificial light radiation is generated using a device chosen from arc lamps, fluorescent lamps, incandescent lamps, light-emitting diodes (LED), organic light-emitting diodes (OLED), or lasers.

39. A method for preparing a composition (B′), wherein the method comprises:
mixing the following ingredients to obtain a mixture:
i) at least one compound of formula (I), 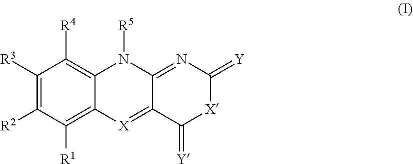
ii) at least one polymerizable molecule chosen from compounds of formula (II), (IIa), (IIb) or (IIc) below, 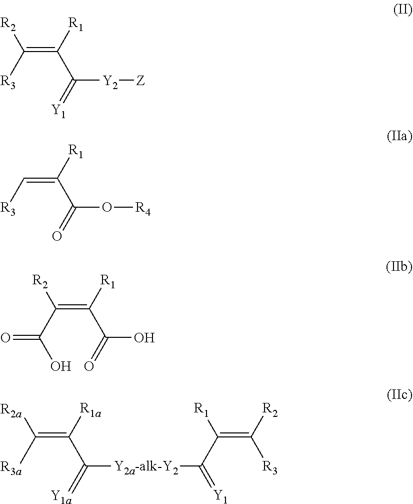
and
iii) optionally, at least one amine and/or at least one alcohol other than i) the compounds of formula (I), and other than ii) the at least one polymerizable molecule; and
exposing the mixture to an artificial or natural light radiation for at least 2 minutes;
wherein in formula (I):
R¹, R², R³ and R⁴, which may be identical or different, are chosen from a hydrogen atom, a halogen atom, or a group chosen from hydroxyl, (C₁-C₆)alkyl, (C₁-C₆)alkoxy, (C₁-C₆)alkylthio, (di)(C₁-C₆)(alkyl)amino, nitro(so);
R⁵ is chosen from a hydrogen atom or a (C₁-C₈)alkyl group optionally substituted with a group chosen from i) hydroxyl, ii) R⁶—C(Y″)—O—, wherein R⁶ is chosen from a hydrogen atom, a (C₁-C₄)alkyl group, or an aryl(C₁-C₄)alkyl group, and Y″ is chosen from an oxygen atom, a sulfur atom, or N(R⁷), wherein R⁷ is chosen from a hydrogen atom or a (C₁-C₄)alkyl group, iii) phosphoric (HO)₂P(O)—O—, iv) —O—[(HO)P(O)—O]ₙ-Suc-Het, wherein Suc is chosen from a divalent sugar group, and Het is chosen from a heteroaryl group, and n is an integer equal to 0, 1, or 2;
X is chosen from a nitrogen atom or a methylene group C(R⁸), wherein R⁸ is chosen from a hydrogen atom or a (C1-C4)alkyl group;
X′ is chosen from an oxygen atom or a group NR9, wherein R9 is chosen from a hydrogen atom or a (C1-C6)alkyl group; and
Y and Y′, which may be identical or different, are chosen from an oxygen atom, a sulfur atom, or a group NR¹⁰, wherein R¹⁰ is chosen from a hydrogen atom or a (C1-C6)alkyl group;
wherein in formulae (II), (IIa), (IIb) and (IIc):
R₁ and R₂, which may be identical or different, are chosen from a hydrogen atom, a (C₁-C₈)alkyl group, or a (C₂-C₈)alkenyl group, wherein the (C₁-C₈)alkyl and the (C₂-C₈)alkenyl groups are:
optionally interrupted with at least one heteroatom chosen from O, S, or N(R₅), wherein R₅ is chosen from a hydrogen atom or a (C₁-C₄)alkyl group, and/or
optionally substituted with at least one radical chosen from the following radicals: hydroxyl, (di)(C₁-C₄)(alkyl)amino, (C₁-C₄)alkoxy, or —C(Y′₁)—Y′₂—R′₄;
R₃ is chosen from a hydrogen atom, a (C₁-C₈)alkyl group, a (C₂-C₈)alkenyl group, or a —C(Y′₁)—Y′₂—R′₄ group;
Y₁, Y₂, Y′₁ and Y′₂, which may be identical or different, are chosen from an oxygen atom, a sulfur atom, or N(R₅), wherein R₅ is chosen from a hydrogen atom or a (C₁-C₄)alkyl group; and
R₄ and R′₄, which may be identical or different, are chosen from a hydrogen atom or a (C₁-C₁₆)alkyl group optionally substituted with at least one radicals, which may be identical or different, chosen from the following radicals: a) amino (NH₂), or mono- or di(C₁-C₄)alkylamino, b) hydroxyl (OH), c) SO₃H, d) carboxyl (CO₂H), e) cyano, f) (poly)(halo)(C₁-C₄)alkyl, g) (C₁-C₄)alkoxycarbonyl; wherein the alkyl radical is optionally interrupted with a heteroatom;
Z is a radical R₄ or a radical (Z′) of formula: 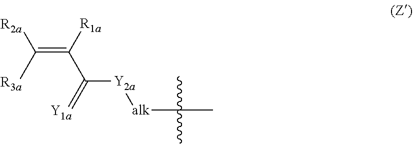
wherein in formula (Z′):
Y₁ₐ, Y₂ₐ, R₁ₐ, R₂ₐ and R₃ₐ have, respectively and independently, the same meanings as that of Y1, Y2, R1, R2 and R3;
alk is a divalent (C₁-C₁₆)alkylene radical optionally interrupted with an oxygen atom, a sulfur atom, or an NH group, the divalent (C₁-C₁₆)alkylene radical being optionally substituted with at least one radical chosen from hydroxyl or a radical of formula (Z″): 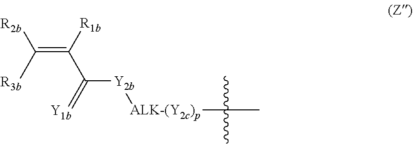
wherein in formula (Z″):
Y_{1b}, Y_{2b}, Y_{2c}, R_{1b}, R_{2b} and R_{3b} have, respectively and independently, the same meanings as that of Y₁, Y₂, R₁, R₂ and R₃; ALK is a (C₁-C₆)alkylene radical (divalent radical); and p is equal to 0 or 1; 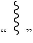
in formula (Z′), the symbol represents the point of attachment between alk and Y₂; and 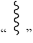
in formula (Z″), the symbol represents the point of attachment between (Y₂)p and ALK.

40. A multi-compartment device comprising:
one or two compartments containing:
a first ingredient i) comprising at least one compound of formula (I), 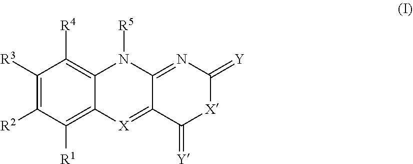
and
a second ingredient ii) comprising at least one polymerizable molecule chosen from compounds of formula (II), (IIa), (IIb) or (IIc); 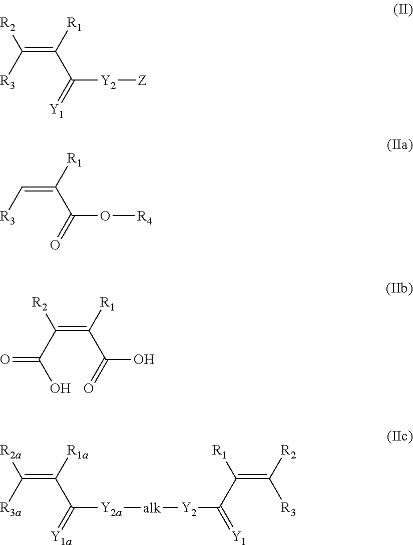
and
a separate compartment comprising an artificial-light-emitting device providing an artificial light radiation with a wavelength ranging from 360 nm to 600 nm;
wherein the first ingredient i) and the second ingredient ii) are contained together in one compartment or separately in two compartments;
wherein in formula (I):
R¹, R², R³ and R⁴, which may be identical or different, are chosen from a hydrogen atom, a halogen atom, or a group chosen from hydroxyl, (C₁-C₆)alkyl, (C₁-C₆)alkoxy, (C₁-C₆)alkylthio, (di)(C₁-C₆)(alkyl)amino, nitro(so);
R⁵ is chosen from a hydrogen atom or a (C₁-C₈)alkyl group optionally substituted with at least one group chosen from i) hydroxyl, ii) R⁶—C(Y″)—O— with R⁶ being chosen from a hydrogen atom, a (C₁-C₄)alkyl group, or an aryl(C₁-C₄)alkyl group, and Y″ is chosen from an oxygen atom, a sulfur atom, or N(R⁷), wherein R⁷ is chosen from a hydrogen atom or a (C₁-C₄)alkyl group, iii) phosphoric (HO)₂P(O)—O—, iv) —O—[(HO)P(O)—O]ₙ-Suc-Het, wherein Suc is chosen from a divalent sugar group, and Het is chosen from a heteroaryl group, n being an integer equal to 0, 1 or 2;
X is chosen from a nitrogen atom or a methylene group C(R8), wherein R8 is chosen from a hydrogen atom or a (C1-C4)alkyl group;
X′ is chosen from an oxygen atom or a group NR9, wherein R9 is chosen from a hydrogen atom or a (C1-C6)alkyl group; and
Y and Y′, which may be identical or different, are chosen from an oxygen atom, a sulfur atom, or a group NR10, wherein R10 is chosen from a hydrogen atom or a (C1-C6)alkyl group; and
in formulae (II), (IIa), (IIb) and (IIc):
R₁ and R₂, which may be identical or different, are chosen from a hydrogen atom, a (C₁-C₈)alkyl group, or a (C₂-C₈)alkenyl group, wherein the (C₁-C₈)alkyl and the (C₂-C₈)alkenyl groups are:
optionally interrupted with at least one heteroatom chosen from 0, S, or N(R₅), wherein R₅ is chosen from a hydrogen atom or a (C₁-C₄)alkyl group, and/or
optionally substituted with at least one radical chosen from the following radicals: hydroxyl, (di)(C₁-C₄)(alkyl)amino, (C₁-C₄)alkoxy or —C(Y′₁)—Y′₂—R′₄;
R3 is chosen from a hydrogen atom, a (C1-C8)alkyl group, a (C2-C8)alkenyl group, or a —C(Y′1)—Y′2—R′4 group;
Y1, Y2, Y′1 and Y′2, which may be identical or different, are chosen from an oxygen atom, a sulfur atom, or N(R5), wherein R5 is chosen from a hydrogen atom or a (C1-C4)alkyl group; and
R4 and R′4, which may be identical or different, are chosen from a hydrogen atom or a (C₁-C₁₆)alkyl group optionally substituted with at least one radical, which may be identical or different, chosen from the following radicals: a) amino (NH₂), or mono- or di(C₁-C₄)alkylamino, b) hydroxyl (OH), c) SO₃H, d) carboxyl (CO₂H), e) cyano, f) (poly)(halo)(C₁-C₄)alkyl, or g) (C₁-C₄)alkoxycarbonyl; wherein the alkyl radical is optionally interrupted with at least one heteroatom;
Z is chosen from a radical R₄ or a radical (Z′) of formula: 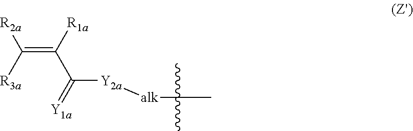
wherein in formula (Z′):
Y1a, Y2a, R1a, R2a and R3a have, respectively and independently, the same meanings as that of Y1, Y2, R1, R2 and R3;
alk is a divalent (C₁-C₁₆)alkylene radical optionally interrupted with an oxygen atom, a sulfur atom, or an NH group, wherein the divalent (C₁-C₁₆)alkylene radical is optionally substituted with one or more radicals chosen from hydroxyl or a radical of formula (Z″): 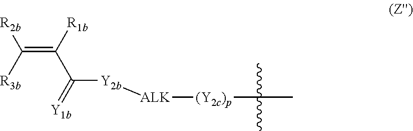
wherein in formula (Z″):
Y_{1b}, Y_{2b}, Y_{2c}, R_{1b}, R_{2b} and R_{3b} have, respectively and independently, the same meanings as that of Y₁, Y₂, R₁, R₂ and R₃, ALK is a (C₁-C₆)alkylene radical (divalent radical), and p is equal to 0 or 1; 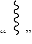
in formula (Z′), the symbol represents the point of attachment between alk and Y₂; and 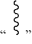
in formula (Z″), the symbol represents the point of attachment between (Y₂)p and ALK.
